(19) United States
(12) Patent Application Publication
(10) Pub. No.: US 2012/0077185 A1
(43) Pub. Date: Mar. 29, 2012

(54) DETECTION OF GENETIC ABNORMALITIES AND INFECTIOUS DISEASE

(75) Inventors: Arnold Oliphant, San Jose, CA (US); Andrew Sparks, San Jose, CA (US); Jacob Zahn, San Jose, CA (US); John Stuelpnagel, San Jose, CA (US); Ken Song, San Jose, CA (US)

(73) Assignee: Tandem Diagnostics, Inc., San Jose, CA (US)

(21) Appl. No.: 13/245,133

(22) Filed: Sep. 26, 2011

Related U.S. Application Data
(63) Continuation-in-part of application No. 13/013,732, filed on Jan. 25, 2011.
Application No. 13/013,732 is a continuation-in-part of application No. 13/205,490, filed on Aug. 8, 2011.
Application No. 13/205,490 is a continuation-in-part of application No. 13/205,570, filed on Aug. 8, 2011.
Application No. 13/205,570 is a continuation-in-part of application No. 13/205,603, filed on Aug. 8, 2011.
(60) Provisional application No. 61/371,605, filed on Aug. 6, 2010.

(51) Int. Cl. C12Q 1/68; C12Q 1/70

(52) U.S. Cl. 435/5; 435/6.12

(57) ABSTRACT

The present invention provides assay systems and related methods for detecting genetic abnormalities and infectious agents in maternal samples. Exemplary maternal samples for analysis using the assay systems of the invention include maternal blood, plasma or serum.

### CROSS-REFERENCE TO RELATED APPLICATIONS

The present application is a continuation-in-part to U.S. Ser. Nos. 13/013,732, filed Jan. 25, 2011; 13/205,490, filed Aug. 8, 2011; 13/205,570, filed Aug. 8, 2011; and 13/205,603, filed Aug. 8, 2011, each of which claim priority to U.S. Ser. No. 61/371,605, filed Aug. 6, 2010, which are all herein incorporated by reference in their entirety.

### FIELD OF THE INVENTION

This invention relates to diagnosis of genetic abnormalities and assay systems for such diagnosis.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Genetic abnormalities account for a wide number of pathologies, including pathologies caused by chromosomal aneuploidy (e.g., Down syndrome), germline mutations in specific genes (e.g., sickle cell anemia), and pathologies caused by somatic mutations (e.g., cancer). Diagnostic methods for determining such genetic anomalies have become standard techniques for identifying specific diseases and disorders, as well as providing valuable information on disease source and treatment options.

For example, prenatal screening and diagnosis are routinely offered in antenatal care and are considered to be important in allowing women to make informed choices about pregnancies affected by genetic conditions. Conventional methods of prenatal diagnostic testing currently requires removal of a sample of fetal cells directly from the uterus for genetic analysis, using either chorionic villus sampling (CVS) typically between 11 and 14 weeks gestation or amniocentesis typically after 15 weeks. However, these invasive procedures carry a risk of miscarriage of around 1%. Mujezinovic and Alfirevic, Obstet Gynecol 2007; 110:687-694.

Although these approaches to obtaining fetal DNA currently provide the gold standard test for prenatal diagnosis, many women decide not to undergo invasive testing, primarily because it is unpleasant and carries a small but significant risk of miscarriage. A reliable and convenient method for non-invasive prenatal diagnosis has long been sought to reduce this risk of miscarriage and allow earlier testing. Although some work has investigated using fetal cells obtained from the cervical mucus (Fejgin M D et al., Prenat Diagn 2001; 21:619-621; Mantzaris et al., ANZJOG 2005; 45:529-532), most research has focused on strategies for detecting genetic elements from the fetus present in the maternal circulation. It has been demonstrated that there is bidirectional traffic between the fetus and the mother during pregnancy (Lo et al., Blood 1996; 88:4390-4395), and multiple studies have shown that both intact fetal cells and cell-free fetal nucleic acids cross the placenta and circulate in the maternal bloodstream (See, e.g., Chiu R W and Lo Y M, Semin Fetal Neonatal Med. 2010 Nov. 11).

In addition, maternal infections can have an adverse impact on fetal health and development, especially during early gestation. Infections affecting the fetus can result in fetal loss or malformations because the ability of the fetus to resist infectious organisms is limited and the fetal immune system is unable to prevent the dissemination of infectious organisms to various tissues. The fetus and/or neonate are infected predominantly by viral but also by bacterial and protozoal pathogens. Infections with various pathogens cause miscarriage or may lead to congenital anomalies in the fetus while others are associated with neonatal infectious morbidity. Identification and treatment of infections with intrauterine and other pathogens in both mother and fetus is thus also an important part of prenatal care.

There is thus a need for non-invasive methods of screening for genetic abnormalities, including aneuploidies, and identifying infectious agents in maternal samples. The present invention addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention provides assay systems and related methods for determining the presence or absence of genetic abnormalities (e.g., chromosomal aneuploidies) and the presence or absence of infectious agents in maternal sample. More specifically, the assay system of the present invention provides the detection of presence or absence of chromosomal abnormalities and detection of presence or absence of one or more infections agents from a single maternal sample. Exemplary maternal samples for analysis using the assay systems of the invention include maternal blood, white blood cells, serum, and plasma.

In a general aspect, the invention provides an assay system for detection of the presence or absence of a chromosomal abnormality and the presence or absence of an infectious agent in a maternal sample, comprising the steps of providing a maternal sample; amplifying two or more selected nucleic acid regions from cell free nucleic acids corresponding to a first chromosome in the maternal sample; amplifying two or more selected nucleic acid regions from cell free nucleic acids corresponding to a second chromosome; amplifying one or more selected nucleic acid regions corresponding to an infectious agent in the maternal sample; detecting the amplified nucleic acid regions; quantifying the relative frequency of the selected nucleic acid regions from the first and second chromosomes; comparing the relative frequency of the selected nucleic acid regions from the first and second chromosomes; identifying the presence or absence of a chromosomal abnormality based on the compared relative frequencies of the first and second chromosomes; and identifying the presence or absence of the infectious agent based on the detected amplified nucleic acid region corresponding to the infectious agent.

In a more specific aspect, the invention provides an assay system for detection of the presence or absence of chromosomal abnormality and the presence or absence of an infectious agent in a maternal sample, comprising the steps of providing a maternal sample; isolating nucleic acids from the maternal sample; amplifying two or more selected nucleic acid regions corresponding to a first chromosome from the isolated nucleic acids; amplifying two or more selected nucleic acid regions corresponding to a second chromosome from the isolated nucleic acids; amplifying one or more selected nucleic acid regions corresponding to an infectious agent from the isolated nucleic acids; detecting the amplified nucleic acid regions; quantifying the relative frequency of the selected nucleic acid regions from the first and second chromosomes; comparing the relative frequency of the selected nucleic acid regions from the first and second chromosomes; identifying the presence or absence of a chromosomal abnormality based on the compared relative frequencies of the first and second chromosome; and identifying the presence or absence of the infectious agent based on the detected amplified nucleic acid region corresponding to the infectious agent.

In preferred aspects, the presence or absence of the genetic abnormality is detected using cell free DNA within the maternal sample as a template for amplification. The amplification template for the detection of the infectious agent may also be cell free nucleic acids, but in certain aspects the template may be nucleic acids obtained from a cellular source (e.g., bacterial or maternal cells) or non-cellular source (e.g., viral particles) present within the maternal sample.

Preferably, the maternal sample used in the assay system of the invention is a single sample that can be utilized for both detection of the chromosomal abnormality and detection of the infectious agent. In some aspects, however, it may be preferable to use separate samples for detection of the chromosomal abnormality and detection of the infectious agent, e.g., when processing of the nucleic acid template requires different isolation conditions.

In certain aspects, the assay format allows the detection of a combination of abnormalities using different detection mechanisms for the nucleic acids corresponding to the chromosomes and the nucleic acids corresponding to the infectious agent. For example, fetal aneuploidy can be determined through the identification and comparison of frequency of selected target nucleic acids in a maternal sample, and the presence or absence of an infectious agent can be identified using non-quantitative mechanisms that are geared towards measuring just the presence or absence of the infectious agent. In other aspects, both the amplified nucleic acids corresponding to the chromosomes and the amplified nucleic acids corresponding to the infectious agent are measured using quantitative detection methods, and more preferably using the same quantitative detection mechanisms (e.g., sequence determination).

In some aspects, the relative frequencies of the selected nucleic acid regions from the chromosomes and the infectious agent are individually quantified, and the relative frequencies of the individual nucleic acid regions corresponding to the chromosomes are compared to determine the presence or absence of the chromosomal abnormality in the mother and/or the fetus. In a more preferred aspect, the relative frequencies of the selected nucleic acid regions are individually quantified and summed by genomic region, and the summations compared to determine the presence or absence of a fetal chromosomal abnormality.

In a more particular aspect, the relative frequencies of the selected nucleic acid regions are individually quantified, and the relative frequencies of the individual nucleic acid regions corresponding to the chromosomes are compared to a reference to determine the presence or absence of a chromosomal aneuploidy in the fetus. In another specific aspect the quantified relative frequencies of the selected nucleic acid regions corresponding to the chromosomes are normalized following detection and prior to quantification.

The infectious agents that can be detected using the assay systems of the invention include, but are not limited to, protozoans, fungi, bacteria and virus. In certain aspects, the nucleic acids corresponding to the presence of an infectious agent in the maternal sample are isolated from the maternal sample prior to analysis. The nucleic acids used in analysis of the infectious agent can be obtained directly from a cell free fraction of the sample, or they may be obtained by processing of cells and/or virions present within the sample.

In some aspects, the nucleic acids isolated from the maternal sample comprise DNA. In other aspects, the nucleic acids isolated from the maternal sample comprise RNA. In preferred aspects, the RNA is optionally converted to DNA prior to amplification of the selected nucleic acid regions. In other aspects, the cell free nucleic acids isolated from the maternal sample comprise both DNA and RNA, and the RNA is optionally converted to DNA prior to the amplification of the selected nucleic acid region.

In some aspects, the nucleic acids from the maternal sample that are used as amplification templates in the assay system of the invention comprise cell free DNA from maternal and fetal sources. In other aspects, nucleic acids from the maternal sample used as amplification templates in the assay system of the invention comprise DNA from a cell source, e.g., a maternal cell, a protozoan cell or a bacterial cell. In yet another aspect, the nucleic acids from the maternal sample used as amplification templates in the assay system of the invention comprise viral DNA, which may be cell free, within a cell or within a virion.

In one preferred aspect the assay utilizes a universal amplification of the selected nucleic acids prior to detection. In another preferred aspect the selected nucleic acid regions are assayed in a single vessel. In yet another preferred aspect, the nucleic acid regions are each counted an average of at least 500 times.

The maternal sample used may be any maternal sample that is obtained using relatively non-invasive means, e.g., maternal blood, maternal plasma or maternal serum. Preferably, the maternal sample is maternal serum.

In one aspect, the assay system utilizes isolation and detection of selected nucleic acid regions in cell free nucleic acids in a maternal sample to identify the presence or absence of a chromosomal abnormality and the presence or absence of an infectious agent. For determination of chromosomal levels, a statistically significant number of selected nucleic acid regions can be determined.

Thus, in a more specific aspect, the invention provides an assay system for detection of the presence or absence of a chromosomal aneuploidy and the presence or absence of an infectious agent in a maternal sample, comprising the steps of providing a maternal sample; amplifying two or more selected nucleic acid regions from cell free nucleic acids corresponding to a first chromosome in the maternal sample; amplifying two or more selected nucleic acid regions from cell free nucleic acids corresponding to a second chromosome; amplifying one or more selected nucleic acid regions corresponding to an infectious agent in the maternal sample; detecting the amplified nucleic acid regions; quantifying the relative frequency of the selected nucleic acid regions from the first and second chromosomes; comparing the relative frequency of the selected nucleic acid regions from the first and second chromosomes; identifying the presence or absence of a chromosomal aneuploidy based on the compared relative frequencies of the first and second chromosome; and identifying the presence or absence of the infectious agent based on the detected amplified nucleic acid region corresponding to the infectious agent.

In a preferred aspect, the invention provides an assay system for detection of the presence or absence of a chromosomal aneuploidy and the presence or absence of an infectious agent in a maternal sample, comprising the steps of providing a maternal sample; isolating nucleic acids from the maternal sample; amplifying two or more selected nucleic acid regions corresponding to a first chromosome from the isolated nucleic acids; amplifying two or more selected nucleic acid regions corresponding to a second chromosome from the isolated nucleic acids; amplifying one or more selected nucleic acid regions corresponding to an infectious agent from the isolated nucleic acids; detecting the amplified nucleic acid regions; quantifying the relative frequency of the selected nucleic acid regions from the first and second chromosomes; comparing the relative frequency of the selected nucleic acid regions from the first and second chromosomes; identifying the presence or absence of a chromosomal aneuploidy based on the compared relative frequencies of the first and second chromosome; and identifying the presence or absence of the infectious agent based on the detected amplified nucleic acid region corresponding to the infectious agent.

In other general aspects, copy number levels of a genomic region of interest can be determined and compared to the quantities of nucleic acid regions of one or more other genomic regions of interest and/or one or more reference genomic regions to detect potential copy number variations. Thus, in another general aspect, the invention provides an assay system for detection of a copy number variation in a genomic region and the presence or absence of an infectious agent in a maternal sample, comprising the steps of providing a maternal sample; amplifying two or more selected nucleic acid regions from cell free nucleic acids corresponding to a first genomic region of interest in the maternal sample; amplifying two or more selected nucleic acid regions from cell free nucleic acids corresponding to a second genomic region of interest in the maternal sample; amplifying one or more selected nucleic acid regions corresponding to an infectious agent in the maternal sample; detecting the amplified nucleic acid regions; quantifying the relative frequency of the selected nucleic acid regions from the first and second genomic regions of interest; comparing the relative frequency of the selected nucleic acid regions from the first and second genomic regions of interest; identifying the presence or absence of a copy number variation based on the compared relative frequencies of the first and second genomic regions of interest; and identifying the presence or absence of the infectious agent based on the detected amplified nucleic acid region corresponding to the infectious agent.

In a preferred aspect, the invention provides an assay system for detection of the presence or absence of a copy number variation and the presence or absence of an infectious agent in a maternal sample, comprising the steps of providing a maternal sample; isolating nucleic acids from the maternal sample; amplifying two or more selected nucleic acid regions corresponding to a first genomic region of interest from the isolated nucleic acids; amplifying two or more selected nucleic acid regions corresponding to a second genomic region of interest from the isolated nucleic acids; amplifying one or more selected nucleic acid regions corresponding to an infectious agent from the isolated nucleic acids; detecting the amplified nucleic acid regions; quantifying the relative frequency of the selected nucleic acid regions from the first and second genomic regions of interest; comparing the relative frequency of the selected nucleic acid regions from the first and second genomic regions of interest; identifying the presence or absence of a copy number variation based on the compared relative frequencies of the first and second genomic regions of interest; and identifying the presence or absence of the infectious agent based on the detected amplified nucleic acid region corresponding to the infectious agent.

In a preferred aspect of the invention, sequences complementary to primers for use in universal amplification are introduced to the selected nucleic acid regions during or following selective amplification. Preferably such sequences are introduced to the ends of such selected nucleic acids, although they may be introduced in any location that allows identification of the amplification product from the universal amplification procedure.

In one general aspect, the assay system utilizes detection of selected regions in cell free DNA in a maternal sample to identify the presence or absence of a copy number variation in a genomic region of interest. In one more specific aspect, the assay system utilizes detection of selected regions in cell free DNA in a maternal sample to identify the presence or absence of a chromosomal aneuploidy. The quantities of selected nucleic acid regions can be determined for a genomic region of interest and compared to the quantities of selected nucleic acid regions from another genomic region of interest and/or to the quantities of selected nucleic acid regions from a reference genomic region of interest to detect potential aneuploidies based on chromosome frequencies in the maternal sample.

In a particular aspect, the ratio of the frequencies of the nucleic acid are compared to a reference mean ratio that has been determined for a statistically significant population of genetically “normal” subjects, i.e. subjects that do not have the particular genetic anomaly that is being interrogated in a particular assay system.

In a preferred aspect, the invention provides an assay system that provides simultaneous detection of the presence or absence of a chromosomal abnormality and the presence or absence of an infectious agent using detection of nucleic acids from a single maternal sample. Preferably, the assay system the nucleic acids are detected in a single vessel, and more preferably the nucleic acids are amplified using universal amplification prior to detection. The nucleic acids are generally isolated from the maternal sample prior to detection. In some aspects, the nucleic acids used for detection of the chromosomal abnormality are isolated separately from the nucleic acids used for the detection of the infectious agent. In such aspects, the isolated nucleic acids are optionally combined prior to detection.

It is a feature of the present invention that the nucleic acid regions are optionally detected using non-polymorphic detection methods, i.e., detection methods that are not dependent upon the presence or absence of a particular polymorphism to identify the selected nucleic acid region. In a preferred aspect, the assay detection systems utilize non-polymorphic detection methods to “count” the relative numbers of selected nucleic acid regions present in a maternal sample. These numbers can be utilized to determine if, statistically, a maternal sample is likely to have a copy number variation in a genomic region. Similarly, these numbers can be utilized to determine if, statistically, one of the DNA origins of a maternal sample is likely to have an abnormal copy number polymorphism. Such information can be used to identify a particular pathology or genetic disorder, to confirm a diagnosis or recurrence of a disease or disorder, to determine the prognosis of a disease or disorder, and/or to assist in determining potential treatment options.

In some aspects, the relative frequencies of selected nucleic acid regions from different chromosomes in a sample are individually quantified and compared to determine the presence or absence of an aneuploidy in a maternal sample. The individually-quantified regions may undergo a normalization calculation or the data may be subjected to outlier exclusion prior to comparison to determine the presence or absence of an aneuploidy in a maternal sample. In other aspects, the relative frequencies of the selected nucleic acid regions are used to determine a chromosome frequency of the first and second chromosomes, and the presence or absence of an aneuploidy is based on the compared chromosome frequencies of the first and second chromosomes. In yet other aspects, the relative frequencies of the selected nucleic acid regions are used to determine a chromosome frequency of a chromosome and a reference chromosome, and the presence or absence of an aneuploidy is based on the compared chromosome frequencies of the chromosome and the reference chromosome.

The assay system of the invention can be configured as a highly multiplexed system which allows for multiple nucleic acid regions from a single or multiple chromosomes within an individual sample and/or multiple samples to be analyzed simultaneously. In such multiplexed systems, the samples can be analyzed separately, or they may be initially pooled into groups of two or more for analysis of larger numbers of samples. When pooled data is obtained, such data is preferably identified for the different samples prior to analysis of aneuploidy. In some aspects, however, the pooled data may be analyzed for potential aneuploidies, and individual samples from the group subsequently analyzed if initial results indicates that a potential aneuploidy is detected within the pooled group.

In some aspects, the nucleic acids in the maternal sample used as templates for the detection of the chromosomal abnormalities are isolated using different methods from the isolation of the nucleic acids used as templates for identification of the infectious agent. In some preferred aspects, the maternal sample may be processed using two distinct isolation mechanisms, and the isolated nucleic acids can either be analyzed separately or preferably the isolated nucleic acids can be recombined and analyzed together for the identification of the chromosomal abnormalities and the presence or absence of infectious agents.

In certain aspects, the assay systems utilize one or more indices that provide information on specific samples or loci. For example, a primer that is used in selective amplification may have additional sequences that are specific to the locus, e.g., a nucleic acid tag sequence that is indicative of the selected nucleic acid region or a particular allele of that nucleic acid region. In another example, an index is used in selective or universal amplification that is indicative of a sample from which the nucleic acid was amplified. In yet another example, a unique identification index is used to distinguish a particular amplification product from other amplification products obtained from the detection methods. A single index may also be combined with any other index to create one index that provides information for two properties (e.g., sample-identification index, allele-locus index).

In one particular aspect, the method of the invention generally comprises detection of the number of copies of two or more selected nucleic acid regions on a first chromosome and two or more selected nucleic acid regions corresponding to a second chromosome, and comparison of the quantities of the selected nucleic acids in a maternal sample to identify the presence or absence of fetal aneuploidy. The selected nucleic acid regions can be isolated from the maternal sample using any means that selectively isolate the particular nucleic acids present in the maternal sample for analysis, e.g., hybridization, amplification or other form of sequence-based isolation of the nucleic acids from the maternal sample. Following isolation, the selected target nucleic acids are individually distributed in a suitable detection format, e.g., on a microarray or in a flow cell, for determination of the relative quantities of each selected nucleic acid in the maternal sample. The relative quantities of the detected nucleic acids are indicative of the number of copies of chromosomes that correspond to the target nucleic acids present in the maternal sample.

Following isolation and distribution of the target nucleic acids in a suitable format, the target sequences are identified, e.g., through sequence determination of the target sequence itself via detection of an associated index (e.g., an identification index, a locus index, an allele index and the like), or via sequence determination and detection of an associated index.

It is a feature of the invention that the nucleic acids analyzed in the assay system do not require polymorphic differences between the fetal and maternal sequences to determine potential aneuploidy. It is another feature of the invention that the substantial majority of the nucleic acids isolated from the maternal sample and detected in the assay system provide information relevant to the presence and quantity of a particular chromosome in the maternal sample, i.e. the detected target nucleic acids are indicative of a particular nucleic acid region associated with a chromosome. This ensures that the majority of nucleic acids analyzed in the assay system of the invention are informative.

In some aspects, multiple nucleic acid regions are determined for each chromosome, and the quantity of the selected regions present in the maternal sample are individually summed to determine the relative frequency of a nucleic acid region in a maternal sample. This includes determination of the frequency of the nucleic acid region for the combined maternal and fetal DNA present in the maternal sample. Preferably, the determination does not require a distinction between the maternal and fetal DNA, although in certain aspects this information may be obtained in addition to the information of relative frequencies in the sample as a whole.

In preferred aspects, target nucleic acids corresponding to multiple nucleic acid regions from a chromosome are detected and summed to determine the relative frequency of a chromosome in the maternal sample. Frequencies that are higher or lower than expected for a nucleic acid region corresponding to one chromosome when compared to the quantity of a nucleic acid region corresponding to another chromosome in the maternal sample are indicative of a fetal aneuploidy. This can be comparison of chromosomes that each may be a putative aneuploid in the fetus (e.g., chromosomes 18 and 21), where the likelihood of both being aneuploid is minimal. This can also be a comparison of chromosomes where one is putatively aneuploid (e.g., chromosome 21) and the other acts as a reference chromosome (e.g. an autosome such as chromosome 2). In yet other aspects, the comparison may utilize two or more chromosomes that are putatively aneuploid and one or more reference chromosomes.

In one aspect, the assay system of the invention analyzes multiple nucleic acids representing selected loci on each chromosome, and the relative frequency of each selected locus from the sample is analyzed to determine a relative chromosome frequency for each particular chromosome in the sample. The chromosomal frequency of two or more chromosomes is then compared to statistically determine whether a chromosomal abnormality exists.

In another aspect, the assay system of the invention analyzes multiple nucleic acids representing selected loci on each chromosome, and the relative frequency of each selected nucleic acid from the sample is analyzed and independently quantified to determine a relative amount for each selected locus in the sample. The sum of the loci in the sample is compared to statistically determine whether a chromosomal aneuploidy exists.

In another aspect, subsets of loci on each chromosome are analyzed to determine whether a chromosomal abnormality exists. The loci frequency can be summed for a particular chromosome, and the summations of the loci used to determine chromosomal abnormalities, e.g., aneuploidy. This aspect of the invention sums the frequencies of the individual loci on each chromosome and then compares the sum of the loci on one chromosome against another chromosome to determine whether a chromosomal abnormality exists. The subsets of loci can be chosen randomly but with sufficient numbers of loci to yield a statistically significant result in determining whether a chromosomal abnormality exists. Multiple analyses of different subsets of loci can be performed within a maternal sample to yield more statistical power. In another aspect, particular loci can be selected on each chromosome that are known to have less variation between maternal samples, or by limiting the data used for determination of chromosomal frequency, e.g., by ignoring the data from loci with very high or very low frequency within a sample.

In a particular aspect, the measured quantity of one or more particular loci on a chromosome is normalized to account for differences in loci quantity in the sample. This can be done by normalizing for known variation from sources such as the assay system (e.g. temperature, reagent lot differences), underlying biology of the sample (e.g. nucleic acid content), operator differences, or any other variables.

In certain specific aspects, determining the relative percentage of fetal DNA in a maternal sample may be beneficial in performing the assays, as it provides important information on the relative statistical presence of nucleic acid regions that may be indicative of fetal aneuploidy. In each maternally-derived sample, the fetus will have 50% of its loci inherited from the mother and 50% of the loci inherited from the father when no copy number variant is present for that locus. Identifying the loci contributed to the fetus from non-maternal sources (e.g., through identification of Y-specific sequences or polymorphisms) allows the estimation of fetal DNA in a maternal sample, thus providing information used to calculate the statistically significant differences in chromosomal frequencies for chromosomes. Such loci thus potentially provides two forms of information in the assay—allelic information can be used to determine the percent fetal DNA contribution in a maternal sample and a summation of the allelic information can be used to determine the relative overall frequency of that locus in a maternal sample. The allelic information is not needed to determine the relative overall frequency of that locus.

Thus, in some specific aspects, the relative contribution of maternal DNA at the allele of interest can be compared to the non-maternal contribution at that allele to determine approximate fetal DNA concentration in the sample. In a particular aspect, the estimation of fetal DNA in a maternal sample is determined at those loci where the mother is homozygous at the locus for a given allele and a different allele is assumed to be inherited by the fetus from the father at that locus. In this situation, the fetal DNA amount will approximately be twice the relative amount of the fetal allele inherited from the father. In other specific aspects, the relative quantity of solely paternally-derived sequences (e.g., Y-chromosome sequences or paternally-specific polymorphisms) can be used to determine the relative concentration of fetal DNA in a maternal sample.

In a specific aspect, the assay system of the invention can be utilized to determine if one or more fetus in a multiples pregnancy is likely to have an aneuploidy, and whether further confirmatory tests should be undertaken to confirm the identification of the fetus with the abnormality. For example, the assay system of the invention can be used to determine if one of two twins has a high likelihood of an aneuploidy, followed by a more invasive technique that can distinguish physically between the fetuses, such as amniocentesis or chorionic villus sampling, to determine the identification of the affected fetus.

In another specific aspect, the assay system of the invention can be utilized to determine if a fetus has a potential mosaicism, and whether further confirmatory tests should be undertaken to confirm the identification of mosaicism in the fetus. Mosaicism could be subsequently confirmed using other testing methods that could distinguish mosaic aneuploidy in specific cells or tissue, either prenatally or postnatally.

In another aspect, the oligonucleotides for a given target nucleic acid can be connected at the non-sequence specific ends such that a circular or unimolecular probe may bind thereto. In this aspect, the 3′ end and the 5′ end of the circular probe binds to the target sequence and at least one universal amplification region is present in the non-target specific sequence of the circular probe.

In some aspects, the assay system is used for both the detection of the presence or absence of fetal aneuploidy and determination of maternal and or fetal carrier status for an allele of interest. Such assays comprise the steps of providing a maternal sample comprising cell free DNA, amplifying two or more selected nucleic acid regions from a first chromosome in the maternal sample, amplifying two or more selected nucleic acid regions from a second chromosome in the maternal sample, amplifying one or more selected nucleic acid regions comprising an allele of interest in the maternal sample, detecting the amplified nucleic acid regions, quantifying the relative frequency of the selected nucleic acid regions from the first and second chromosomes, comparing the relative frequency of the selected nucleic acid regions from the first and second chromosomes, and identifying the presence or absence of a genetic alteration based on the compared relative frequencies of the first and second chromosome.

In these assay systems, relative frequencies of the selected nucleic acid regions can be individually quantified, and the relative frequencies of the individual nucleic acid regions compared to determine the presence or absence of a genetic alteration in the fetal or maternal nucleic acids. Such quantified relative frequencies of the selected nucleic acid regions are optionally normalized following detection and prior to quantification.

In preferred aspects, the nucleic acid regions are assayed in a single vessel, and the nucleic acid regions undergo a universal amplification. In other preferred aspects, the selected nucleic acid regions are each counted an average of at least 500 times.

These and other aspects, features and advantages will be provided in more detail as described herein.

### DETAILED DESCRIPTION OF THE INVENTION

The methods described herein may employ, unless otherwise indicated, conventional techniques and descriptions of molecular biology (including recombinant techniques), cell biology, biochemistry, and microarray and sequencing technology, which are within the skill of those who practice in the art. Such conventional techniques include polymer array synthesis, hybridization and ligation of oligonucleotides, sequencing of oligonucleotides, and detection of hybridization using a label. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W.H. Freeman, New York (1995); Gait, “Oligonucleotide Synthesis: A Practical Approach” IRL Press, London (1984); Nelson and Cox, Lehninger, Principles of Biochemistry, 3^{rd} Ed., W. H. Freeman Pub., New York (2000); and Berg et al., Biochemistry, 5^{th} Ed., W.H. Freeman Pub., New York (2002), all of which are herein incorporated by reference in their entirety for all purposes. Before the present compositions, research tools and methods are described, it is to be understood that this invention is not limited to the specific methods, compositions, targets and uses described, as such may, of course, vary. It is also to be understood that the terminology used herein is for the purpose of describing particular aspects only and is not intended to limit the scope of the present invention, which will be limited only by appended claims.

It should be noted that as used herein and in the appended claims, the singular forms “a,” “and,” and “the” include plural referents unless the context clearly dictates otherwise. Thus, for example, reference to “a nucleic acid region” refers to one, more than one, or mixtures of such regions, and reference to “an assay” includes reference to equivalent steps and methods known to those skilled in the art, and so forth.

Where a range of values is provided, it is to be understood that each intervening value between the upper and lower limit of that range—and any other stated or intervening value in that stated range—is encompassed within the invention. Where the stated range includes upper and lower limits, ranges excluding either of those included limits are also included in the invention.

Unless expressly stated, the terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated. All publications mentioned herein are incorporated by reference for the purpose of describing and disclosing the formulations and methodologies that are described in the publication and which might be used in connection with the presently described invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “amplified nucleic acid” is any nucleic acid molecule whose amount has been increased at least two fold by any nucleic acid amplification or replication method performed in vitro as compared to its starting amount in a maternal sample.

The term “chromosomal abnormality” refers to any genetic variant for all or part of a chromosome. The genetic variants may include but not be limited to any copy number variants such as duplications or deletions, translocations, inversions, and mutations.

The terms “complementary” or “complementarity” are used in reference to nucleic acid molecules (i.e., a sequence of nucleotides) that are related by base-pairing rules. Complementary nucleotides are, generally, A and T (or A and U), or C and G. Two single stranded RNA or DNA molecules are said to be substantially complementary when the nucleotides of one strand, optimally aligned and with appropriate nucleotide insertions or deletions, pair with at least about 90% to about 95% complementarity, and more preferably from about 98% to about 100% complementarity, and even more preferably with 100% complementarity. Alternatively, substantial complementarity exists when an RNA or DNA strand will hybridize under selective hybridization conditions to its complement. Selective hybridization conditions include, but are not limited to, stringent hybridization conditions. Stringent hybridization conditions will typically include salt concentrations of less than about 1 M, more usually less than about 500 mM and preferably less than about 200 mM. Hybridization temperatures are generally at least about 2° C. to about 6° C. lower than melting temperatures (Tₘ).

The term “correction index” refers to an index that may contain additional nucleotides that allow for identification and correction of amplification, sequencing or other experimental errors including the detection of deletion, substitution, or insertion of one or more bases during sequencing as well as nucleotide changes that may occur outside of sequencing such as oligo synthesis, amplification, and any other aspect of the assay. These correction indices may be stand-alone indices that are separate sequences, or they may be embedded within other indices to assist in confirming accuracy of the experimental techniques used, e.g., a correction index may be a subset of sequences of a locus index or an identification index.

The term “corresponding to” as used herein refers to a nucleic acid that is indicative of a chromosome or infectious agent, as the case may be. For example, a nucleic acid corresponding to a chromosome or a region thereof may be found on that chromosome, in the case of a subchromosomal region, adjacent to a genomic region within a chromosome. In the case of an infectious agent, the nucleic acid may be a genetic component (DNA or RNA) from the infectious agent itself or it may be a nucleic acid that is produced by the host in response to a particular infectious agent.

The term “diagnostic tool” as used herein refers to any composition or assay of the invention used in combination as, for example, in a system in order to carry out a diagnostic test or assay on a patient sample.

The term “hybridization” generally means the reaction by which the pairing of complementary strands of nucleic acid occurs. DNA is usually double-stranded, and when the strands are separated they will re-hybridize under the appropriate conditions. Hybrids can form between DNA-DNA, DNA-RNA or RNA-RNA. They can form between a short strand and a long strand containing a region complementary to the short one. Imperfect hybrids can also form, but the more imperfect they are, the less stable they will be (and the less likely to form).

The term “identification index” refers generally to a series of nucleotides incorporated into a primer region of an amplification process for unique identification of an amplification product of a nucleic acid region. Identification index sequences are preferably 6 or more nucleotides in length. In a preferred aspect, the identification index is long enough to have statistical probability of labeling each molecule with a target sequence uniquely. For example, if there are 3000 copies of a particular target sequence, there are substantially more than 3000 identification indexes such that each copy of a particular target sequence is likely to be labeled with a unique identification index. The identification index may contain additional nucleotides that allow for identification and correction of sequencing errors including the detection of deletion, substitution, or insertion of one or more bases during sequencing as well as nucleotide changes that may occur outside of sequencing such as oligo synthesis, amplification, and any other aspect of the assay. The index may be combined with any other index to create one index that provides information for two properties (e.g. sample-identification index, locus-identification index).

The terms “locus” and “loci” as used herein refer to a nucleic acid region of known location in a genome.

The term “locus index” refers generally to a series of nucleotides that correspond to a known locus on a chromosome. Generally, the locus index is long enough to label each known locus region uniquely. For instance, if the method uses 192 known locus regions corresponding to 192 individual sequences associated with the known loci, there are at least 192 unique locus indexes, each uniquely identifying a region indicative of a particular locus on a chromosome. The locus indices used in the methods of the invention may be indicative of different loci on a single chromosome as well as known loci present on different chromosomes within a sample. The locus index may contain additional nucleotides that allow for identification and correction of sequencing errors including the detection of deletion, substitution, or insertion of one or more bases during sequencing as well as nucleotide changes that may occur outside of sequencing such as oligo synthesis, amplification, and any other aspect of the assay.

The term “maternal sample” as used herein refers to any sample taken from a pregnant mammal which comprises both fetal and maternal cell free genomic material (e.g., DNA). Preferably, maternal samples for use in the invention are obtained through relatively non-invasive means, e.g., phlebotomy or other standard techniques for extracting peripheral samples from a subject.

The term “melting temperature” or Tₘ is commonly defined as the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+16.6(log 10[Na+])0.41(%[G+C])−675/n−1.0m, when a nucleic acid is in aqueous solution having cation concentrations of 0.5 M or less, the (G+C) content is between 30% and 70%, n is the number of bases, and m is the percentage of base pair mismatches (see, e.g., Sambrook J et al., Molecular Cloning, A Laboratory Manual, 3rd Ed., Cold Spring Harbor Laboratory Press (2001)). Other references include more sophisticated computations, which take structural as well as sequence characteristics into account for the calculation of Tₘ.

“Microarray” or “array” refers to a solid phase support having a surface, preferably but not exclusively a planar or substantially planar surface, which carries an array of sites containing nucleic acids such that each site of the array comprises substantially identical or identical copies of oligonucleotides or polynucleotides and is spatially defined and not overlapping with other member sites of the array; that is, the sites are spatially discrete. The array or microarray can also comprise a non-planar interrogatable structure with a surface such as a bead or a well. The oligonucleotides or polynucleotides of the array may be covalently bound to the solid support, or may be non-covalently bound. Conventional microarray technology is reviewed in, e.g., Schena, Ed., Microarrays: A Practical Approach, IRL Press, Oxford (2000). “Array analysis”, “analysis by array” or “analysis by microarray” refers to analysis, such as, e.g., sequence analysis, of one or more biological molecules using a microarray.

By “non-polymorphic”, when used with respect to detection of selected nucleic acid regions, is meant a detection of such nucleic acid region, which may contain one or more polymorphisms, but in which the detection is not reliant on detection of the specific polymorphism within the region. Thus a selected nucleic acid region may contain a polymorphism, but detection of the region using the assay system of the invention is based on occurrence of the region rather than the presence or absence of a particular polymorphism in that region.

As used herein “nucleotide” refers to a base-sugar-phosphate combination. Nucleotides are monomeric units of a nucleic acid sequence (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP.

According to the present invention, a “nucleotide” may be unlabeled or detectably labeled by well known techniques. Fluorescent labels and their attachment to oligonucleotides are described in many reviews, including Haugland, Handbook of Fluorescent Probes and Research Chemicals, 9th Ed., Molecular Probes, Inc., Eugene Oreg. (2002); Keller and Manak, DNA Probes, 2nd Ed., Stockton Press, New York (1993); Eckstein, Ed., Oligonucleotides and Analogues: A Practical Approach, IRL Press, Oxford (1991); Wetmur, Critical Reviews in Biochemistry and Molecular Biology, 26:227-259 (1991); and the like. Other methodologies applicable to the invention are disclosed in the following sample of references: Fung et al., U.S. Pat. No. 4,757,141; Hobbs, Jr., et al., U.S. Pat. No. 5,151,507; Cruickshank, U.S. Pat. No. 5,091,519; Menchen et al., U.S. Pat. No. 5,188,934; Begot et al., U.S. Pat. No. 5,366,860; Lee et al., U.S. Pat. No. 5,847,162; Khanna et al., U.S. Pat. No. 4,318,846; Lee et al., U.S. Pat. No. 5,800,996; Lee et al., U.S. Pat. No. 5,066,580: Mathies et al., U.S. Pat. No. 5,688,648; and the like. Labeling can also be carried out with quantum dots, as disclosed in the following patents and patent publications: U.S. Pat. Nos. 6,322,901; 6,576,291; 6,423,551; 6,251,303; 6,319,426; 6,426,513; 6,444,143; 5,990,479; 6,207,392; 2002/0045045; and 2003/0017264. Detectable labels include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′ dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg.

The terms “oligonucleotides” or “oligos” as used herein refer to linear oligomers of natural or modified nucleic acid monomers, including deoxyribonucleotides, ribonucleotides, anomeric forms thereof, peptide nucleic acid monomers (PNAs), locked nucleotide acid monomers (LNA), and the like, or a combination thereof, capable of specifically binding to a single-stranded polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g., 8-12, to several tens of monomeric units, e.g., 100-200 or more. Suitable nucleic acid molecules may be prepared by the phosphoramidite method described by Beaucage and Carruthers (Tetrahedron Lett., 22:1859-1862 (1981)), or by the triester method according to Matteucci, et al. (J. Am. Chem. Soc., 103:3185 (1981)), both incorporated herein by reference, or by other chemical methods such as using a commercial automated oligonucleotide synthesizer.

As used herein the term “polymerase” refers to an enzyme that links individual nucleotides together into a long strand, using another strand as a template. There are two general types of polymerase—DNA polymerases, which synthesize DNA, and RNA polymerases, which synthesize RNA. Within these two classes, there are numerous sub-types of polymerases, depending on what type of nucleic acid can function as template and what type of nucleic acid is formed.

As used herein “polymerase chain reaction” or “PCR” refers to a technique for replicating a specific piece of target DNA in vitro, even in the presence of excess non-specific DNA. Primers are added to the target DNA, where the primers initiate the copying of the target DNA using nucleotides and, typically, Taq polymerase or the like. By cycling the temperature, the target DNA is repetitively denatured and copied. A single copy of the target DNA, even if mixed in with other, random DNA, can be amplified to obtain billions of replicates. The polymerase chain reaction can be used to detect and measure very small amounts of DNA and to create customized pieces of DNA. In some instances, linear amplification methods may be used as an alternative to PCR.

The term “polymorphism” as used herein refers to any genetic changes in a locus that may be indicative of that particular locus, including but not limited to single nucleotide polymorphisms (SNPs), methylation differences, short tandem repeats (STRs), and the like.

Generally, a “primer” is an oligonucleotide used to, e.g., prime DNA extension, ligation and/or synthesis, such as in the synthesis step of the polymerase chain reaction or in the primer extension techniques used in certain sequencing reactions. A primer may also be used in hybridization techniques as a means to provide complementarity of a nucleic acid region to a capture oligonucleotide for detection of a specific nucleic acid region.

The term “research tool” as used herein refers to any composition or assay of the invention used for scientific enquiry, academic or commercial in nature, including the development of pharmaceutical and/or biological therapeutics. The research tools of the invention are not intended to be therapeutic or to be subject to regulatory approval; rather, the research tools of the invention are intended to facilitate research and aid in such development activities, including any activities performed with the intention to produce information to support a regulatory submission.

The term “sample index” refers generally to a series of unique nucleotides (i.e., each sample index is unique to a sample in a multiplexed assay system for analysis of multiple samples). The sample index can thus be used to assist in nucleic acid region identification for multiplexing of different samples in a single reaction vessel, such that each sample can be identified based on its sample index. In a preferred aspect, there is a unique sample index for each sample in a set of samples, and the samples are pooled during sequencing. For example, if twelve samples are pooled into a single sequencing reaction, there are at least twelve unique sample indexes such that each sample is labeled uniquely. The index may be combined with any other index to create one index that provides information for two properties (e.g., sample-identification index, sample-locus index).

The term “selected nucleic acid region” as used herein refers to a nucleic acid region corresponding to an individual chromosome. Such selected nucleic acid regions may be directly isolated from the sample for detection, e.g., based on hybridization and/or other sequence-based techniques, or they may be amplified using the sample as a template prior to detection of the sequence. Nucleic acid regions for use in the assay systems of the present invention may be selected on the basis of DNA level variation between individuals, based upon specificity for a particular chromosome, based on CG content and/or required amplification conditions of the selected nucleic acid regions, or other characteristics that will be apparent to one skilled in the art upon reading the present disclosure.

The terms “sequencing”, “sequence determination” and the like as used herein refers generally to any and all biochemical methods that may be used to determine the order of nucleotide bases in a nucleic acid.

The term “specifically binds”, “specific binding” and the like as used herein, when referring to a binding partner (e.g., a nucleic acid probe or primer, antibody, etc.) that results in the generation of a statistically significant positive signal under the designated assay conditions. Typically the interaction will subsequently result in a detectable signal that is at least twice the standard deviation of any signal generated as a result of undesired interactions (background).

### The Invention in General

The present invention provides improved methods for identifying copy number variants (CNVs) of particular genomic regions, including complete chromosomes (e.g., aneuploidies), in maternal samples as well as the presence or absence of an infectious agent. The methods to detect CNVs and/or other chromosomal abnormalities are not reliant upon the presence or absence of any polymorphic or mutation information, and thus are conceptually agnostic as to the genetic variation that may be present in any chromosomal region under interrogation. These methods are useful for any maternal sample containing cell free genomic material (e.g., DNA) from two or more cell types of interest, e.g., maternal samples comprising maternal and fetal cell free DNA, maternal samples comprising cell free DNA from normal and putatively malignant cells, maternal samples comprising cell free DNA from a transplant donor and recipient, maternal samples comprising cell free DNA from any maternal cell and from an infectious agent, and the like.

The assay methods of the invention provide in addition to methods for detecting one or more infectious agents, isolation and amplification of nucleic acid regions from chromosomes of interest and/or reference chromosomes for copy number variant detection. A distinct advantage of the invention is that the selected nucleic acid regions can be analyzed using a variety of detection and quantification techniques, including but not limited to hybridization techniques, digital PCR and high throughput sequencing determination techniques. Selection probes can be designed against any number of nucleic acid regions for any chromosome and for the one or more infectious agents. Although amplification prior to the identification and quantification of the selection nucleic acids regions is not mandatory, limited amplification prior to detection is preferred.

The present invention provides an improved system over more random techniques such as massively parallel sequencing, shotgun sequencing, and the use of random digital PCR which have been used by others to detect copy number variations in maternal samples such as maternal blood. These aforementioned approaches rely upon sequencing of all or a statistically significant population of DNA fragments in a sample, followed by mapping of these fragments or otherwise associating the fragments to their appropriate chromosomes. The identified fragments are then compared against each other or against some other reference (e.g. normal chromosomal makeup) to determine copy number variation of particular chromosomes. These methods are inherently inefficient from the present invention, as the primary chromosomes of interest only constitute a minority of data that is generated from the detection of such DNA fragments in the maternal samples.

Techniques that are dependent upon a very broad sampling of DNA in a sample are providing a very broad coverage of the DNA analyzed, but in fact are sampling the DNA contained within a sample on a 1× or less basis (i.e., subsampling). In contrast, the selective amplification used in the present assays are specifically designed to provide depth of coverage of particular nucleic acids of interest, and provide a “super-sampling” of such selected regions with an average sequence coverage of preferably 2× or more, more preferably sequence coverage of 100× of more, even more preferably sequence coverage of 1000× or more of the selected nucleic acids present in the initial maternal sample.

The methods of the invention provide a more efficient and economical use of data, and the substantial majority of sequences analyzed following sample amplification result in affirmative information about the presence of a particular chromosome and/or infectious agent(s) in the sample. Thus, unlike techniques relying on massively parallel sequencing or random digital “counting” of chromosome regions and subsequent identification of relevant data from such counts, the assay system of the invention provides a much more efficient use of data collection than the random approaches taught by others in the art.

The sequences analyzed using the assay system of the present invention are amplified representative sequences selected from various regions of the chromosomes of interest to determine the relative quantity of the chromosomes in the maternal sample, and the substantial majority of sequences analyzed are informative of the presence of a region on a chromosome of interest and/or a reference chromosome. These techniques do not require the analysis of large numbers of sequences which are not from the chromosomes of interest and which do not provide information on the relative quantity of the chromosomes of interest.

### Detecting Chromosomal Aneuploidies

The present invention provides methods for identifying fetal chromosomal aneuploidies in maternal samples comprising both maternal and fetal DNA. Such methods can be performed using amplification methods for identification of nucleic acid regions corresponding to specific chromosomes of interest and/or reference chromosomes in the maternal sample.

The assay systems utilize nucleic acid probes designed to identify, and preferably to isolate, selected nucleic acids regions in a maternal sample that correspond to individual chromosomes of interest and, in certain aspects, to reference chromosomes that are used to determine the presence or absence of aneuploidy in a maternal sample. These probes are specifically designed to hybridize to a selected nucleic acid region of a particular chromosome, and thus quantification of the nucleic acid regions in a maternal sample using these probes is indicative of the copy number of a particular chromosome in the maternal sample.

In preferred aspects, the assay systems of the invention employ one or more selective amplification steps (e.g., using one or more primers that specifically hybridize to a selected nucleic acid region) to enhance the DNA content of a sample and/or to provide improved mechanisms for isolating, amplifying or analyzing the selected nucleic acid regions. This is in direct contrast to the random amplification approach used by others employing, e.g., massively parallel sequencing, as such amplification techniques generally involve random amplification of all or a substantial portion of the genome.

In a general aspect, the user of the invention analyzes multiple target sequences on different chromosomes and determines the frequency or amount of the target sequences of the chromosomes simultaneously. When multiple target sequences are analyzed on chromosomes, a preferred embodiment is to amplify all of the target sequences for each sample in one reaction vessel. The frequency or amount of the multiple target sequences on the different chromosomes is then compared to determine whether a chromosomal abnormality exists.

In one aspect, the user of the invention analyzes multiple target sequences on multiple chromosomes and averages the frequency of the target sequences on the multiple chromosomes together. Normalization or standardization of the frequencies can be performed for one or more target sequences.

In another aspect, the user of the invention sums the frequencies of the target sequences on each chromosome and then compares the sum of the target sequences on one chromosome against another chromosome to determine whether a chromosomal abnormality exists. In another aspect, one analyzes subsets of target sequences on each chromosome to determine whether a chromosomal abnormality exists. The comparison can be made either within the same or different chromosomes.

In certain aspects, the data used to determine the frequency of the target sequences may exclude outlier data that appear to be due to experimental error, or that have elevated or depressed levels based on an idiopathic genetic bias within a particular sample. In one example, the data used for summation may exclude DNA regions with a particularly elevated frequency in one or more samples. In another example, the data used for summation may exclude target sequences that are found in a particularly low abundance in one or more samples.

In another aspect subsets of loci can be chosen randomly but with sufficient numbers of loci to yield a statistically significant result in determining whether a chromosomal abnormality exists. Multiple analyses of different subsets of loci can be performed within a maternal sample to yield more statistical power. For example, if there are 100 selected regions for chromosome 21 and 100 selected regions for chromosome 18, a series of analyses could be performed that evaluate fewer than 100 regions for each of the chromosomes. In this example, target sequences are not being selectively excluded.

The quantity of different nucleic acids detectable on certain chromosomes may vary depending upon a number of factors, including general representation of fetal loci in maternal samples, degradation rates of the different nucleic acids representing fetal loci in maternal samples, sample preparation methods, and the like. Thus, in another aspect, the quantity of particular loci on a chromosome is summed to determine the loci quantity for different chromosomes in the sample. The loci frequency is summed for a particular chromosome, and the sum of the loci are used to determine aneuploidy. This aspect of the invention sums the frequencies of the individual loci on each chromosome and then compares the sum of the loci on one chromosome against another chromosome to determine whether a chromosomal abnormality exists.

The nucleic acids analyzed using the assay systems of the invention are preferably selectively amplified and optionally isolated from the maternal sample using primers specific to the nucleic acid region of interest (e.g., to a locus of interest in a maternal sample). The primers for such selective amplification designed to isolate regions may be chosen for various reasons, but are preferably designed to 1) efficiently amplify a region from the chromosome of interest; 2) have a predictable range of expression from maternal and/or fetal sources in different maternal samples; 3) be distinctive to the particular chromosome, i.e., not amplify homologous regions on other chromosomes. The following are exemplary techniques that may be employed in the assay system or the invention.

### Detection of Infectious Agents in Maternal Samples

The assay system of the invention is designed to identify the presence or absence of infectious agents as well as detecting chromosomal abnormalities using a single maternal sample. Detection of exogenous agents in a maternal sample may be indicative of exposure to and infection by an infectious agent, and this finding have an impact on patient care or management of an infectious disease for which a subject tests positively for such infectious agent.

In fact, pregnancy itself may be a risk factor for acquiring certain infectious diseases, such as toxoplasmosis, Hansen disease, and listeriosis. In addition, for pregnant women or subjects with suppressed immune systems, certain infectious diseases such as influenza and varicella may have a more severe clinical course, increased complication rate, and higher case-fatality rate. Identification of infectious disease agents may therefore allow better treatment for maternal disease during pregnancy, leading to a better overall outcome for both mother and fetus.

The detection of the infectious agent may be performed in a simultaneous fashion with the detection of the chromosomal abnormality e.g., through detection of cell free nucleic acids indicative of the infectious agent in the maternal sample, or the nucleic acids corresponding to the infectious agent may be detected following a different analytical mechanism, e.g., the isolated of nucleic acids from cells or virions within the maternal sample.

In addition, certain infectious agents can be passed to the fetus via vertical transmission, i.e. spread of infections from mother to baby. These infections may occur while the fetus is still in the uterus, during labor and delivery, or after delivery (such as while breastfeeding).

Thus, in some preferred aspects, the assay system includes detection of exogenous sequences, e.g., sequences from infectious organisms that may have an adverse effect on the health and/or viability of the fetus or infant, in order to protect maternal, fetal, and or infant health.

Exemplary infections which can be spread via vertical transmission, and which can be tested for using the assay methods of the invention, include but are not limited to congenital infections, perinatal infections and postnatal infections.

Congenital infections are passed in utero by crossing the placenta to infect the fetus. Many infectious microbes can cause congenital infections, leading to problems in fetal development or even death. TORCH is an acronym for several of the more common congenital infections. These are: toxoplasmosis, other infections (e.g., syphilis, hepatitis B, Coxsackie virus, Epstein-Barr virus, varicella-zoster virus (chicken pox), and human parvovirus B19 (fifth disease)), rubella, cytomegalovirus (CMV), and herpes simplex virus.

Perinatal infections refer to infections that occur as the baby moves through an infected birth canal or through contamination with fecal matter during delivery. These infections can include, but are not limited to, sexually-transmitted diseases (e.g., gonorrhea, chlamydia, herpes simplex virus, human papilloma virus, etc.) CMV, and Group B Streptococci (GBS).

Infections spread from mother to baby following delivery are known as postnatal infections. These infections can be spread during breastfeeding through infectious microbes found in the mother's breast milk. Some examples of postnatal infections are CMV, Human immunodeficiency virus (HIV), Hepatitis C Virus (HCV), and GBS.

A more comprehensive list of potential infectious agents that can be measured using the assay systems of the invention are as set forth in Tables 1-3. Table 1 lists exemplary non-viral infectious agents that can be detected using the assay systems of the invention. Table 2 lists the exemplary DNA virus that can be detected using the assay systems of the invention. Table 3 lists the exemplary RNA virus that can be detected using the assay systems of the invention. These tables are not meant to be exhaustive lists, and it will be apparent to one skilled in the art upon reading the present disclosure that the assay systems of the invention are useful for other similar infectious organisms that may be present in a maternal sample.

TABLE 1
Prokaryotic and Eukaryotic Infectious Agents

| Disease | Infectious Agent | Nature of Pathogen |
|---|---|---|
| Brucellosis, | Brucella melitensis | Gram negative bacteria |
| Bang's disease | | |
| Candidiasis/ | Candida albicans | Fungus |
| candidemia | (or other yeast strains) | |
| Chagas | Trypanosma cruzi | Trypanosome |
| Chlamydia | Chlamydia trachomatis | RNA/DNA bacterium |
| Food poisoning | Escheria coli O157:H7 | Gram negative bacteria |
| Lyme disease | Borrelia burgdorferi | Spirochete bacterium |
| Leishmaniasis, | Leishmania chagasi | Protozoan |
| kala-azar | (infantum) | |
| Leptospirosis/ | Leptospira Spp. | Spirochete bacterium |
| Weil's disease | | |
| Listeriosis | Listeria monocytogenes | Gram positive bacterium |
| Malaria | Plasmodium species | Eukaryotic Protist |
| Meningitis | Neisseria meningitides | Gram positive bacterium |
| Pertussis | Bordetella pertussis | Gram negative bacterium |
| (Whooping Cough) | | |
| Rocky Mountain | Rickettsia rickettsii | Gram negative bacterium |
| Spotted Fever | | |
| Syphilis | Treponema pallidum | Spirochete bacterium |
| Toxoplasmosis | Toxoplamsa gondi | Protozoan |

TABLE 2
DNA virus

| Disease | Infectious Agent | Nature of Pathogen |
|---|---|---|
| Acute respiratory | Adenovirus | ssDNA |
| disease | | (Adenoviridae) |
| Chicken pox/ | varicella zoster virus | dsDNA virus |
| shingles | | (Herpesviridae) |
| Fifth disease | Parvovirus B19 | ssDNA virus |
| | | (Parvoviridae) |
| Genital warts/ | Human Papilloma virus | dsDNA virus |
| cervical cancer | | (Papillomaviridae) |
| Herpes simplex | HSV-1 and HSV-2 | dsDNA virus |
| | | (Herpesviridae) |
| Hepatitis B | Human Hepatitis B | dsDNA virus |
| | virus (HBV) | (Hepadnavirus) |
| Hepatitis B | Human Hepatitis B | dsDNA virus |
| | virus (HBV) | (Hepadnavirus) |
| Human Cyto- | human herpesvirus-5 | dsDNA virus |
| megalovirus | (HHV-5) | (Herpesviridae) |
| Infection | | |
| Mononucleosis/ | Epstein Barr virus | dsDNA virus |
| various cancers | (HHV-4) | (Herpesviridae) |

TABLE 3
RNA virus

| Disease | Infectious Agent | Nature of Pathogen |
|---|---|---|
| Aseptic meningitis | Lymphocytic chorio- | ssRNA |
| | meningitis virus (LCMV) | (Arenaviridae) |
| Dengue fever | Dengue virus | ssRNA |
| | | (Flaviviridae) |
| Gastroenteritis | Rotavirus | dsRNA |
| | | (Reoviridae) |
| Gastroenteritis | Norovirus | ssRNA |
| | | (Caliciviridae) |
| Hantavirus Pulmonary | Hantavirus (Sin Nombre | ssRNA Virus |
| Syndrome | virus) | (Bunyaviridae) |
| Hepatitis A | Human hepatitis A virus | ssRNA |
| | | (Picornaviridae) |
| Hepatitis C | Human Hepatitis C | ssRNA virus |
| | virus (HCV) | (Hepaciiridae) |
| Hepatitis D | Human Hepatitis D | ssRNA virus |
| | virus (HDV) | (Deltairidae) |
| Hepatitis E | Human Hepatitis E | ssRNA virus |
| | virus (HEV) | (Hepesiridae) |
| HIV/AIDS | Human immuno- | dsRNA virus |
| | deficiency virus-1 | (Retroviridae) |
| Influenza | Influenza virus | ssRNA |
| | | (Orthomyxoviridae) |
| Measles | Morbillivirus | ssRNA |
| | | (Paramyxoviridae) |
| Mumps (Parotitis) | Rubulavirus | ssRNA |
| | | (Paramyxoviridae) |
| Neonatal sepsis | Enterovirus | ssRNA |
| | | (Picornaviridae) |
| Poliomyelitis | Polio Virus | ssRNA |
| | | (Picornaviridae) |
| Rubella (German | Rubella Virus | ssRNA |
| Measles) | | (Togaviridae) |
| Swine flu | H1N1 virus | ssRNA |
| | | (Orthomyxoviridae) |
| West Nile | West Nile virus | ssRNA |
| Encephalitis | | (Flaviviridae) |
| Yellow Fever | Yellow Fever virus | ssRNA |
| | | (Flaviviridae) |

For the DNA-based pathogens, such as those listed in Tables 1 and 2, the assays utilize a straightforward method of detecting the infectious by direct amplification of DNA from the infectious organism isolated from a maternal sample, with or without an initial isolation step from the initial maternal sample. For RNA virus, such as those listed in Table 3, although direct amplification of RNA can be employed in certain aspects, it is generally preferred that DNA be used as an amplification template as RNA is very easily degraded by RNases. Detection of DNA from an RNA virus can be generally can be performed two ways: 1) by using a DNA intermediate that is part of the viral lifecycle as a template for amplification of selected pathogen loci using the assays of the invention, or 2) by isolating the RNA from the virus and converting it biochemically to complementary DNA (cDNA), which is then used as a template for amplification of the selected pathogen loci using the assays of the invention.

Many RNA virus replicate directly via RNA intermediates and utilize an RNA-dependent RNA-polymerase to replicate their RNA. For example, the virion of plus-stranded RNA viruses (e.g., poliovirus and flavivirus) comprises plus-stranded RNA, which can function directly as an mRNA for purposes of protein production. The virions of a negative sense RNA virus (e.g., orthomyxovirus or paramyxovirus) are converted to a plus-sense mRNA but does not go through a DNA intermediate. Yet other exemplary double-stranded RNA viruses (e.g., picorna virus) also use the RNA to produce an mRNA without conversion to DNA. To detect these viruses, the assays systems of the invention preferably utilize the isolation of the RNA molecules (plus- or negative-stranded) and conversion to a cDNA prior to the amplification of the selected loci.

Other RNA viruses, and in particular the retrovirus, comprise a plus-sense RNA virion that is copied into DNA. This DNA is incorporated into the host genome during the latent phase of the virus. For these RNA virus, infection can be identified through use of the DNA intermediate in the genome as a template for amplification of selected nucleic acids. In certain aspects, however, even if a virus life cycle does comprise a DNA intermediate, the detection of these viruses is preferably performed by creation of a cDNA prior to amplification of selected loci, as this may be more indicative of an active viral infection as opposed to the latent phase of the virus in the maternal genome.

Bacterial genomes are mosaic structures composed of genes present in every strain of the same species (i.e., the core genome), and genes present in some but not all strains of a species (i.e., the accessory genome). Accessory genome genes include, e.g., antibiotic resistance genes and virulence plasmids. In certain preferred aspects, the detection of nucleic acids associated with bacterial pathogens using the methods of the invention can include the detection of the nucleic acids from the accessory genome as well as detection of nucleic acids in the core genome. This allows the detection of the strain of bacteria and the genes involved in pathogenesis and antibiotic resistance from the accessory genome, which can inform treatment options and lead to better clinical outcomes for the mother and fetus.

In specific aspects, the assay systems of the invention can comprise multiple reactions that interrogate the fetal aneuploidy, presence or absence of infectious disease pathogens, detection of other agents or risk factors, and the maternal and/or fetal carrier status in a single reaction. Combining all of these forms of screening requires only one blood draw and is both more efficient and more cost effective, as the incremental costs of adding additional tests is much less than testing conditions separately.

Isolation of Nucleic Acids from the Maternal Sample

In certain aspects, the nucleic acid regions that are detected in the assay systems of the invention are amplified from nucleic acids isolated from the maternal sample. The nucleic acids from the infectious agents are optionally isolated from the maternal sample prior to the analysis using the assay system of the invention.

Exemplary methods for isolating nucleic acids from a maternal sample can be found at U.S. Pat. Nos. 7,989,614; 7,727,727; 7,601,491; 7,527,929; 6,914,137; 6,562,573; 6,534,262; 6,342,387; 6,310,199; 5,898,071; 5,808,041; 20100184210; 20040214175; and J Clin Microbiol. 1991 March; 29(3): 422-425. Viral DNA and/or RNA can be isolated from a maternal sample using techniques well know to those skilled in the art (See, e.g., PureLink™ Pro 96 Viral RNA/DNA Purification Kit or Tempus™ Spin RNA Isolation Kit (Life Technologies, Carlsbad, Calif.).

When RNA is isolated, it may be converted to DNA using one of three general mechanisms. In certain aspects in which the mRNA to be interrogated has a poly-A 3′ tail, an oligo-dT primer can be used to prime the mRNAs. In other aspects, sequence-specific primers can be employed which will selectively convert those RNA of interest. Third, random primer can be utilized to produce shorter DNA fragments and with an increased probability that 5′ ends of the RNA are converted to cDNA.

With certain infectious agents, there is also a threshold level required for the presence of the infection becomes clinically relevant. For example, low levels of some infectious agents such as serotype of CMV may be present in a sample but not at a level that would cause concern of a significant infection leading to a clinical outcome. In addition, certain sequences may be present in a maternal sample from a previous infection that is no longer clinically relevant e.g., low levels of an infectious agent that are indicative of a latent infection (e.g., presence of DNA from a herpes virus or a retrovirus) may not be clinically significant enough to suggest there is an active infection that may affect the fetus.

Thus, in certain aspects, the detected levels of infectious agent must be compared to a reference level or reference sample. Such reference samples may be spiked with the nucleic acids from the infectious agent at levels that are generally present in a latent stage, that are present in levels that are indicative of a clinically relevant infection that may impact on maternal and/or fetal health, and the like. The reporting of the presence or absence of an infectious disease would thus be based on the identification of a clinically relevant level of the infectious disease rather than merely the presence of the infectious agent in a sample.

### Selected Amplification

Numerous selective amplification methods can be used to provide the amplified nucleic acids that are analyzed in the assay systems of the invention, and such methods are preferably used to increase the copy numbers of a nucleic acid region of interest in a maternal sample in a manner that allows preservation of information concerning the initial content of the nucleic acid region in the maternal sample. Although not all combinations of amplification and analysis are described herein in detail, it is well within the skill of those in the art to utilize different amplification methods and/or analytic tools to isolate and/or analyze the nucleic acids of region consistent with this specification, and such variations will be apparent to one skilled in the art upon reading the present disclosure.

Such amplification methods include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, 1989; Landegren et al., Science 241:1077, 1988), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NASBA). (See, U.S. Pat. Nos. 5,409,818, 5,554,517, and 6,063,603, each of which is incorporated herein by reference). Other amplification methods that may be used include: Qbeta Replicase, described in PCT Patent Application No. PCT/US87/00880, isothermal amplification methods such as SDA, described in Walker et al. 1992, Nucleic Acids Res. 20(7):1691-6, 1992, and rolling circle amplification, described in U.S. Pat. No. 5,648,245. Other amplification methods that may be used are described in, U.S. Pat. Nos. 5,242,794, 5,494,810, 4,988,617 and in U.S. Ser. No. 09/854,317 and US Pub. No. 20030143599, each of which is incorporated herein by reference. In some aspects DNA is amplified by multiplex locus-specific PCR. In a preferred aspect the DNA is amplified using adaptor-ligation and single primer PCR. Other available methods of amplification, such as balanced PCR (Makrigiorgos, et al. (2002), Nat Biotechnol, Vol. 20, pp. 936-9) and isothermal amplification methods such as nucleic acid sequence based amplification (NASBA) and self-sustained sequence replication (Guatelli et al., Proc. Natl. Acad. Sci. USA 87: 1874, 1990) may be employed.). Based on such methodologies, a person skilled in the art can readily design primers in any suitable regions 5′ and 3′ to a nucleic acid region of interest. Such primers may be used to amplify DNA of any length so long that it contains the nucleic acid region of interest in its sequence.

The length of an amplified selected nucleic acid from a genomic region of interest is generally long enough to provide enough sequence information to distinguish it from other nucleic acids that are amplified and/or selected. Generally, an amplified nucleic acid is at least about 16 nucleotides in length, and more typically, an amplified nucleic acid is at least about 20 nucleotides in length. In a preferred aspect of the invention, an amplified nucleic acid is at least about 30 nucleotides in length. In a more preferred aspect of the invention, an amplified nucleic acid is at least about 32, 40, 45, 50, or 60 nucleotides in length. In other aspects of the invention, an amplified nucleic acid can be about 100, 150 or up to 200 in length.

In certain aspects, the selected amplification comprises an initial linear amplification step. This can be particularly useful if the starting amount of DNA is quite limited, e.g., where the cell-free DNA in a sample is available in limited quantities. This mechanism increases the amount of DNA molecules that are representative of the original DNA content, and helps to reduce sampling error where accurate quantification of the DNA or a fraction of the DNA (e.g., fetal DNA contribution in a maternal sample) is needed.

Thus, in one aspect, a limited number of cycles of sequence-specific linear amplification are performed on the starting maternal sample comprising cell free DNA. The number of cycles is generally less than that used for a typical PCR amplification, e.g., 5-30 cycles or fewer. Primers or probes may be designed to amplify specific genomic segments or regions. The primers or probes may be modified with an end label at the 5′ end (e.g. with biotin) or elsewhere along the primer or probe such that the amplification products could be purified or attached to a solid substrate (e.g., bead or array) for further isolation or analysis. In a preferred aspect, the primers are multiplexed such that a single reaction yields multiple DNA fragments from different regions. Amplification products from the linear amplification can then be further amplified with standard PCR methods or with additional linear amplification.

For example, cell free DNA can be isolated from blood, plasma, or serum from a pregnant woman, and incubated with primers against a set number of nucleic acid regions that correspond to chromosomes of interest. Preferably, the number of primers used for initial linear amplification will be 12 or more, more preferably 24 or more, more preferably 36 or more, even more preferably 48 or more, and even more preferably 96 or more. Each of the primers corresponds to a single nucleic acid region, and is optionally tagged for identification and/or isolation. A limited number of cycles, preferably 10 or fewer, are performed with linear amplification. The amplification products are subsequently isolated, e.g., when the primers are linked to a biotin molecule the amplification products can be isolated via binding to avidin or streptavidin on a solid substrate. The products are then subjected to further biochemical processes such as further amplification with other primers and/or detection techniques such as sequence determination and hybridization.

Efficiencies of linear amplification may vary between sites and between cycles so that in certain systems normalization may be used to ensure that the products from the linear amplification are representative of the nucleic acid content starting material. One practicing the assay system of the invention can utilize information from various samples to determine variation in nucleic acid levels, including variation in different nucleic acid regions in individual samples and/or between the same nucleic acid regions in different samples following the limited initial linear amplification. Such information can be used in normalization to prevent skewing of initial levels of DNA content.

### Universal Amplification

In preferred aspects of the invention, the selectively amplified nucleic acid regions are preferably amplified following selective amplification, either prior to or during the nucleic acid region detection techniques. In another aspect of the invention, nucleic acid regions are selectively amplified during the nucleic acid region detection technique without any prior amplification. In a multiplexed assay system, this is preferably done through universal amplification of the various nucleic acid regions to be analyzed using the assay systems of the invention. Universal primer sequences are added to the selectively amplified nucleic acid regions so that they may be further amplified in a single universal amplification reaction. These universal primer sequences may be added to the nucleic acids regions during the selective amplification process, i.e., the primers for selective amplification have universal primer sequences that flank a locus. Alternatively, adapters comprising universal amplification sequences can be added to the ends of the selected nucleic acids as adapters following amplification and isolation of the selected nucleic acids from the maternal sample.

In one exemplary aspect, nucleic acids are initially amplified from a maternal sample using primers complementary to selected regions of the chromosomes of interest, followed by a universal amplification step to increase the number of nucleic acid regions for analysis. This introduction of primer regions to the initial amplification products from a maternal sample allows a subsequent controlled universal amplification of all or a portion of selected nucleic acids prior to or during analysis, e.g. sequence determination.

Bias and variability can be introduced during DNA amplification, such as that seen during polymerase chain reaction (PCR). In cases where an amplification reaction is multiplexed, there is the potential that loci will amplify at different rates or efficiency. Part of this may be due to the variety of primers in a multiplex reaction with some having better efficiency (i.e. hybridization) than others, or some working better in specific experimental conditions due to the base composition. Each set of primers for a given locus may behave differently based on sequence context of the primer and template DNA, buffer conditions, and other conditions. A universal DNA amplification for a multiplexed assay system will generally introduce less bias and variability.

Accordingly, in a preferred aspect, a small number (e.g., 1-10, preferably 3-5) of cycles of selected amplification in a multiplexed mixture reaction are performed, followed by universal amplification using introduced universal primers. The number of cycles using universal primers will vary, but will preferably be at least 10 cycles, more preferably at least 5 cycles, even more preferably 20 cycles or more. By moving to universal amplification following a lower number of amplification cycles, the bias of having certain loci amplify at greater rates than others is reduced.

Optionally, the assay system will include a step between the selected amplification and universal amplification to remove any excess nucleic acids that are not specifically amplified in the selected amplification.

The whole product or an aliquot of the product from the selected amplification may be used for the universal amplification. The same or different conditions (e.g., polymerase, buffers, and the like) may be used in the amplification steps, e.g., to ensure that bias and variability is not inadvertently introduced due to experimental conditions. In addition, variations in primer concentrations may be used to effectively limit the number of sequence specific amplification cycles.

In certain aspects, the universal primer regions of the primers or adapters used in the assay system are designed to be compatible with conventional multiplexed assay methods that utilize general priming mechanisms to analyze large numbers of nucleic acids simultaneously in one reaction in one vessel. Such “universal” priming methods allow for efficient, high volume analysis of the quantity of nucleic acid regions present in a maternal sample, and allow for comprehensive quantification of the presence of nucleic acid regions within such a maternal sample for the determination of aneuploidy.

Examples of such assay methods include, but are not limited to, multiplexing methods used to amplify and/or genotype a variety of samples simultaneously, such as those described in Oliphant et al., U.S. Pat. No. 7,582,420

Some aspects utilize coupled reactions for multiplex detection of nucleic acid sequences where oligonucleotides from an early phase of each process contain sequences which may be used by oligonucleotides from a later phase of the process. Exemplary processes for amplifying and/or detecting nucleic acids in samples can be used, alone or in combination, including but not limited to the methods described below, each of which are incorporated by reference in their entirety.

In certain aspects, the assay system of the invention utilizes one of the following combined selective and universal amplification techniques: (1) LDR coupled to PCR; (2) primary PCR coupled to secondary PCR coupled to LDR; or (3) primary PCR coupled to secondary PCR. Each of these aspects of the invention has particular applicability in detecting certain nucleic acid characteristics. However, each requires the use of coupled reactions for multiplex detection of nucleic acid sequence differences where oligonucleotides from an early phase of each process contain sequences which may be used by oligonucleotides from a later phase of the process.

Barany et al., U.S. Pat. Nos. 6,852,487, 6,797,470, 6,576,453, 6,534,293, 6,506,594, 6,312,892, 6,268,148, 6,054,564, 6,027,889, 5,830,711, 5,494,810, describe the use of the ligase chain reaction (LCR) assay for the detection of specific sequences of nucleotides in a variety of nucleic acid samples.

Barany et al., U.S. Pat. Nos. 7,807,431, 7,455,965, 7,429,453, 7,364,858, 7,358,048, 7,332,285, 7,320,865, 7,312,039, 7,244,831, 7,198,894, 7,166,434, 7,097,980, 7,083,917, 7,014,994, 6,949,370, 6,852,487, 6,797,470, 6,576,453, 6,534,293, 6,506,594, 6,312,892, and 6,268,148 describe the use of the ligase detection reaction with detection reaction (“LDR”) coupled with polymerase chain reaction (“PCR”) for nucleic acid detection.

Barany et al., U.S. Pat. Nos. 7,556,924 and 6,858,412, describe the use of padlock probes (also called “precircle probes” or “multi-inversion probes”) with coupled ligase detection reaction (“LDR”) and polymerase chain reaction (“PCR”) for nucleic acid detection.

Barany et al., U.S. Pat. Nos. 7,807,431, 7,709,201, and 7,198,814 describe the use of combined endonuclease cleavage and ligation reactions for the detection of nucleic acid sequences.

Willis et al., U.S. Pat. Nos. 7,700,323 and 6,858,412, describe the use of precircle probes in multiplexed nucleic acid amplification, detection and genotyping, including

Ronaghi et al., U.S. Pat. No. 7,622,281 describes amplification techniques for labeling and amplifying a nucleic acid using an adapter comprising a unique primer and a barcode.

In a preferred aspect, the nucleic acid detection used to provide data on selected biomolecules utilizes selected amplification of representative loci. Such techniques are disclosed in, e.g., US Appln Nos. 13/013,732, 13/205,490, 13/205,570, and 13/205,603. These techniques utilize detection of genomic regions using fixed sequence oligonucleotides and joining them via ligation and/or extension. This can be accomplished using a combination of ligation and amplification, e.g., the ligation of two or more fixed sequence oligonucleotides and optionally a bridging oligonucleotide that is complementary to a region between the fixed sequence oligonucleotides.

In addition to the various amplification techniques, numerous methods of sequence determination are compatible with the assay systems of the inventions. Preferably, such methods include “next generation” methods of sequencing. Exemplary methods for sequence determination include, but are not limited to, including, but not limited to, hybridization-based methods, such as disclosed in Drmanac, U.S. Pat. Nos. 6,864,052; 6,309,824; and 6,401,267; and Drmanac et al, U.S. patent publication 2005/0191656, which are incorporated by reference, sequencing by synthesis methods, e.g., Nyren et al, U.S. Pat. Nos. 7,648,824, 7,459,311 and 6,210,891; Balasubramanian, U.S. Pat. Nos. 7,232,656 and 6,833,246; Quake, U.S. Pat. No. 6,911,345; Li et al, Proc. Natl. Acad. Sci., 100: 414-419 (2003); pyrophosphate sequencing as described in Ronaghi et al., U.S. Pat. Nos. 7,648,824, 7,459,311, 6,828,100, and 6,210,891; and ligation-based sequencing determination methods, e.g., Drmanac et al., U.S. Pat. Appln No. 20100105052, and Church et al, U.S. Pat. Appln Nos. 20070207482 and 20090018024.

Alternatively, nucleic acid regions of interest can be selected and/or identified using hybridization techniques. Methods for conducting polynucleotide hybridization assays for detection of have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davis, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred aspects. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

### Use of Indices in the Assay Systems of the Invention

In certain aspects, all or a portion of the sequences of the nucleic acids of interest are directly detected using the described techniques, e.g., sequence determination or hybridization. In certain aspects, however, the nucleic acids of interest are associated with one or more indices that are identifying for a selected nucleic acid region or a particular sample being analyzed. The detection of the one or more indices can serve as a surrogate detection mechanism of the selected nucleic acid region, or as confirmation of the presence of a particular selected nucleic acid region if both the sequence of the index and the sequence of the nucleic acid region itself are determined. These indices are preferably associated with the selected nucleic acids during an amplification step using primers that comprise both the index and sequence regions that specifically hybridize to the nucleic acid region.

In one example, the primers used for amplification of a selected nucleic acid region are designed to provide a locus index between the selected nucleic acid region primer region and a universal amplification region. The locus index is unique for each selected nucleic acid region and representative of a locus on a chromosome of interest or reference chromosome, so that quantification of the locus index in a sample provides quantification data for the locus and the particular chromosome containing the locus.

In another example, the primers used for amplification of a selected nucleic acid region are designed to provide an allele index between the selected nucleic acid region primer region and a universal amplification region. The allele index is unique for particular alleles of a selected nucleic acid region and representative of a locus variation present on a chromosome of interest or reference chromosome, so that quantification of the allele index in a sample provides quantification data for the allele and the summation of the allelic indices for a particular locus provides quantification data for both the locus and the particular chromosome containing the locus.

In another aspect, the primers used for amplification of the selected nucleic acid regions to be analyzed for a maternal sample are designed to provide an identification index between the selected nucleic acid region primer region and a universal amplification region. In such an aspect, a sufficient number of identification indices are present to uniquely identify each selected nucleic acid region in the sample. Each nucleic acid region to be analyzed is associated with a unique identification index, so that the identification index is uniquely associated with the selected nucleic acid region. Quantification of the identification index in a sample provides quantification data for the associated selected nucleic acid region and the chromosome corresponding to the selected nucleic acid region. The identification index may also be used to detect any amplification bias that occurs downstream of the initial isolation of the selected nucleic acid regions from a sample.

In certain aspects, only the locus index and/or the identification index (if present) are detected and used to quantify the selected nucleic acid regions in a sample. In another aspect, a count of the number of times each locus index occurs with a unique identification index is performed to determine the relative frequency of a selected nucleic acid region in a sample.

In some aspects, indices representative of the sample from which a nucleic acid is isolated are used to identify the source of the nucleic acid in a multiplexed assay system. In such aspects, the nucleic acids are uniquely identified with the sample index. Those uniquely identified oligonucleotides may then be combined into a single reaction vessel with nucleic acids from other samples prior to sequencing. The sequencing data is first segregated by each unique sample index prior to determining the frequency of each target locus for each sample and prior to determining whether there is a chromosomal abnormality for each sample. For detection, the sample indices, the locus indices, and the identification indices (if present), are sequenced.

In aspects of the invention using indices, the selective amplification primers are preferably designed so that indices comprising identifying information are coded at one or both ends of the primer. Alternatively, the indices and universal amplification sequences can be added to the selectively amplified nucleic acids following initial amplification.

The indices are non-complementary but unique sequences used within the primer to provide information relevant to the selective nucleic acid region that is isolated and/or amplified using the primer. The advantage of this is that information on the presence and quantity of the selected nucleic acid region can be obtained without the need to determine the actual sequence itself, although in certain aspects it may be desirable to do so. Generally, however, the ability to identify and quantify a selected nucleic acid region through identification of one or more indices will decrease the length of sequencing required as the loci information is captured at the 3′ or 5′ end of the isolated selected nucleic acid region. Use of indices identification as a surrogate for identification of selected nucleic acid regions may also reduce error since longer sequencing reads are more prone to the introduction or error.

In addition to locus indices, allele indices and identification indices, additional indices can be introduced to primers to assist in the multiplexing of samples. For example, correction indices which identify experimental error (e.g., errors introduced during amplification or sequence determination) can be used to identify potential discrepancies in experimental procedures and/or detection methods in the assay systems. The order and placement of these indices, as well as the length of these indices, can vary, and they can be used in various combinations.

The primers used for identification and quantification of a selected nucleic acid region may be associated with regions complementary to the 5′ of the selected nucleic acid region, or in certain amplification regimes the indices may be present on one or both of a set of amplification primers which comprise sequences complementary to the sequences of the selected nucleic acid region. The primers can be used to multiplex the analysis of multiple selected nucleic acid regions to be analyzed within a sample, and can be used either in solution or on a solid substrate, e.g., on a microarray or on a bead. These primers may be used for linear replication or amplification, or they may create circular constructs for further analysis.

Variation Minimization within and Between Samples

One challenge with the detection of chromosomal abnormalities in a maternal sample is that often the DNA from the cell type with the putative chromosomal abnormality is present in much lower abundance than the DNA from normal cell type. In the case of a mixed maternal sample containing fetal and maternal cell free DNA, the cell free fetal DNA as a percentage of the total cell free DNA may vary from less than one to forty percent, and most commonly is present at or below twenty percent and frequently at or below ten percent. In the detection of an aneuploidy such as Trisomy 21 (Down Syndrome) in the fetal DNA of such mixed maternal sample, the relative increase in Chromosome 21 is 50% in the fetal DNA and thus as a percentage of the total DNA in a maternal sample where, as an example, the fetal DNA is 5% of the total, the increase in Chromosome 21 as a percentage of the total is 2.5%. If one is to detect this difference robustly through the methods described herein, the variation in the measurement of Chromosome 21 has to be much less than the percent increase of Chromosome 21.

The variation between levels found between samples and/or for nucleic acid regions within a sample may be minimized in a combination of analytical methods, many of which are described in this application. For instance, variation is lessened by using an internal reference in the assay. An example of an internal reference is the use of a chromosome present in a “normal” abundance (e.g., disomy for an autosome) to compare against a chromosome present in putatively abnormal abundance, such as aneuploidy, in the same sample. While the use of one such “normal” chromosome as a reference chromosome may be sufficient, it is also possible to use many normal chromosomes as the internal reference chromosomes to increase the statistical power of the quantification.

One method of using an internal reference is to calculate a ratio of abundance of the putatively abnormal chromosomes to the abundance of the normal chromosomes in a sample, called a chromosomal ratio. In calculating the chromosomal ratio, the abundance or counts of each of the nucleic acid regions for each chromosome are summed together to calculate the total counts for each chromosome. The total counts for one chromosome are then divided by the total counts for a different chromosome to create a chromosomal ratio for those two chromosomes.

Alternatively, a chromosomal ratio for each chromosome may be calculated by first summing the counts of each of the nucleic acid regions for each chromosome, and then dividing the sum for one chromosome by the total sum for two or more chromosomes. Once calculated, the chromosomal ratio is then compared to the average chromosomal ratio from a normal population.

The average may be the mean, median, mode or other average, with or without normalization and exclusion of outlier data. In a preferred aspect, the mean is used. In developing the data set for the chromosomal ratio from the normal population, the normal variation of the measured chromosomes is calculated. This variation may be expressed a number of ways, most typically as the coefficient of variation, or CV. When the chromosomal ratio from the sample is compared to the average chromosomal ratio from a normal population, if the chromosomal ratio for the sample falls statistically outside of the average chromosomal ratio for the normal population, the sample contains an aneuploidy. The criteria for setting the statistical threshold to declare an aneuploidy depend upon the variation in the measurement of the chromosomal ratio and the acceptable false positive and false negative rates for the desired assay. In general, this threshold may be a multiple of the variation observed in the chromosomal ratio. In one example, this threshold is three or more times the variation of the chromosomal ratio. In another example, it is four or more times the variation of the chromosomal ratio. In another example it is five or more times the variation of the chromosomal ratio. In another example it is six or more times the variation of the chromosomal ratio. In the example above, the chromosomal ratio is determined by summing the counts of nucleic acid regions by chromosome. Typically, the same number of nucleic acid regions for each chromosome is used. An alternative method for generating the chromosomal ratio would be to calculate the average counts for the nucleic acid regions for each chromosome. The average may be any estimate of the mean, median or mode, although typically an average is used. The average may be the mean of all counts or some variation such as a trimmed or weighted average. Once the average counts for each chromosome have been calculated, the average counts for each chromosome may be divided by the other to obtain a chromosomal ratio between two chromosomes, the average counts for each chromosome may be divided by the sum of the averages for all measured chromosomes to obtain a chromosomal ratio for each chromosome as described above. As highlighted above, the ability to detect an aneuploidy in a maternal sample where the putative DNA is in low relative abundance depends greatly on the variation in the measurements of different nucleic acid regions in the assay. Numerous analytical methods can be used which reduce this variation and thus improve the sensitivity of this method to detect aneuploidy. One method for reducing variability of the assay is to increase the number of nucleic acid regions used to calculate the abundance of the chromosomes. In general, if the measured variation of a single nucleic acid region of a chromosome is X % and Y different nucleic acid regions are measured on the same chromosome, the variation of the measurement of the chromosomal abundance calculated by summing or averaging the abundance of each nucleic acid region on that chromosome will be approximately X % divided by Ŷ½. Stated differently, the variation of the measurement of the chromosome abundance would be approximately the average variation of the measurement of each nucleic acid region's abundance divided by the square root of the number of nucleic acid regions.

In a preferred aspect of this invention, the number of nucleic acid regions measured for each chromosome is at least 24. In another preferred aspect of this invention, the number of nucleic acid regions measured for each chromosome is at least 48. In another preferred aspect of this invention, the number of nucleic acid regions measured for each chromosome is at least 100. In another preferred aspect of this invention the number of nucleic acid regions measured for each chromosome is at least 200. There is incremental cost to measuring each nucleic acid region and thus it is important to minimize the number of each nucleic acid region. In a preferred aspect of this invention, the number of nucleic acid regions measured for each chromosome is less than 2000. In a preferred aspect of this invention, the number of nucleic acid regions measured for each chromosome is less than 1000. In a most preferred aspect of this invention, the number of nucleic acid regions measured for each chromosome is at least 48 and less than 1000. In one aspect, following the measurement of abundance for each nucleic acid region, a subset of the nucleic acid regions may be used to determine the presence or absence of aneuploidy. There are many standard methods for choosing the subset of nucleic acid regions. These methods include outlier exclusion, where the nucleic acid regions with detected levels below and/or above a certain percentile are discarded from the analysis. In one aspect, the percentile may be the lowest and highest 5% as measured by abundance. In another aspect, the percentile may be the lowest and highest 10% as measured by abundance. In another aspect, the percentile may be the lowest and highest 25% as measured by abundance.

Another method for choosing the subset of nucleic acid regions includes the elimination of regions that fall outside of some statistical limit. For instance, regions that fall outside of one or more standard deviations of the mean abundance may be removed from the analysis. Another method for choosing the subset of nucleic acid regions may be to compare the relative abundance of a nucleic acid region to the expected abundance of the same nucleic acid region in a healthy population and discard any nucleic acid regions that fail the expectation test. To further minimize the variation in the assay, the number of times each nucleic acid region is measured may be increased. As discussed, in contrast to the random methods of detecting aneuploidy where the genome is measured on average less than once, the assay systems of the present invention intentionally measures each nucleic acid region multiple times. In general, when counting events, the variation in the counting is determined by Poisson statistics, and the counting variation is typically equal to one divided by the square root of the number of counts. In a preferred aspect of the invention, the nucleic acid regions are each measured on average at least 100 times. In a preferred aspect to the invention, the nucleic acid regions are each measured on average at least 500 times. In a preferred aspect to the invention, the nucleic acid regions are each measured on average at least 1000 times. In a preferred aspect to the invention, the nucleic acid regions are each measured on average at least 2000 times. In a preferred aspect to the invention, the nucleic acid regions are each measured on average at least 5000 times.

In another aspect, subsets of loci can be chosen randomly but with sufficient numbers of loci to yield a statistically significant result in determining whether a chromosomal abnormality exists. Multiple analyses of different subsets of loci can be performed within a maternal sample to yield more statistical power. In this example, it may or may not be necessary to remove or eliminate any loci prior to the random analysis. For example, if there are 100 selected regions for chromosome 21 and 100 selected regions for chromosome 18, a series of analyses could be performed that evaluate fewer than 100 regions for each of the chromosomes.

In addition to the methods above for reducing variation in the assay, other analytical techniques, many of which are described earlier in this application, may be used in combination. In general, the variation in the assay may be reduced when all of the nucleic acid regions for each sample are interrogated in a single reaction in a single vessel. Similarly, the variation in the assay may be reduced when a universal amplification system is used. Furthermore, the variation of the assay may be reduced when the number of cycles of amplification is limited.

### Detection of Genetic Mutations

In certain aspects, the assay system of the invention detects both chromosomal abnormalities and specific genetic alterations in specific loci of interest. Such additional genetic alterations include, but are not limited to, deletion mutations, insertion mutations, copy number polymorphisms, copy number variants, chromosome 22q11 deletion syndrome, 11q deletion syndrome on chromosome 11, 8p deletion syndrome on chromosome 8, and the like. Generally, at least two target nucleic acid sequences present on the same or separate chromosomes are analyzed, and at least one of the target sequences is associated with the fetal allelic abnormality. The sequences of the two target sequences and number of copies of the two target sequences are then compared to determine whether the chromosomal abnormality is present, and if so, the nature of the abnormality.

While much of the description contained herein describes detecting aneuploidy by counting the abundance of nucleic acid regions on one or more putative aneuploid chromosomes and the abundance of nucleic acid regions on one or more normal chromosomes, the same techniques may be used to detect copy number variations where such copy number variation occurs on only a portion of a chromosome. In this detection of the copy number variations, multiple nucleic acid regions within the putative copy number variation location are compared to multiple nucleic acid regions outside of the putative copy number variation location. Other aspects of the invention described for aneuploidy may then be used for the detection of copy number variation. For instance, one may detect a chromosome 22q11 deletion syndrome in a fetus in a mixed maternal sample by selecting two or more nucleic regions within the 22q11 deletion and two or more nucleic acid regions outside of the 22q11 deletion. The nucleic acid regions outside of the 22q11 deletion may be on another region of Chromosome 22 or may be on a completely different chromosome. The abundance of each nucleic acid regions is determined by the methods described in this application.

In some aspects a universal amplification may be used for amplifying the nucleic acid regions. In some aspects, the nucleic acid regions for each sample are assayed in a single reaction in a single vessel. In some aspects, at least 24 nucleic acid regions may be used within the deletion and at least 24 nucleic acid regions may be used outside of the deletion. In another aspect at least 48 nucleic acid regions may be used within the deletion and at least 48 nucleic acid regions may be used outside of the deletion. The nucleic acid regions within the deletion are then summed as are the nucleic acid regions outside of the deletion. These sums are then compared to each other to determine the presence or absence of a deletion. Optionally, the sums are put into a ratio and that ratio may be compared to an average ratio created from a normal population. When the ratio for a sample falls statistically outside of an expected ratio, the deletion is detected. The threshold for the detection of a deletion may be four or more times the variation calculated in the normal population.

### Use of Other Fetal Detection Methods in Risk Assessment

In certain aspects of the invention, the methods of the invention can be used in conjunction with detection of other known risk factors (e.g., maternal age, family history, maternal or paternal genetic information) and/or means for detecting fetal abnormalities, and preferably with other relatively non-invasive diagnostic mechanisms of fetal abnormalities (e.g., measurements of one or more biochemical markers in a maternal sample and/or measurements or structural detection from an ultrasound scan). The combined use of these risk factors and diagnostic mechanisms with the methods of the invention can provide an improved risk determination of fetal abnormality, and in particular the presence or absence of a known genetic mutation such as a trisomy.

Thus, in some preferred aspects the results obtained in the assay systems of the invention are combined with the results from biochemical detection of risk factors, ultrasound detection of risk factors, or other risk determinants of fetal abnormalities.

In some specific aspects, the results obtained in the assay systems of the invention are combined with detection of biochemical markers associated with an increased risk of fetal abnormality. The biochemical markers can be determined based on a sample comprising maternal blood, serum, plasma or urine. Such biochemical markers include but are not limited to free Beta hCG, pregnancy-associated plasma protein A (PAPP-A), maternal blood alpha-fetoprotein, maternal blood hCG, maternal blood unconjugated estriol, maternal blood dimeric inhibin A, maternal urine total estriol, maternal urine beta core fragment, maternal urine hyperglycosylated hCG, maternal blood hyperglycosylated hCG, and inhibin A (preferably dimeric inhibin A). In some aspects, the additional assessment mechanism is multimarker analysis, such as that described in Orlandi et al., U.S. Pat. No. 7,315,787 or Wald et al. U.S. Pat. No. 6,573,103. Detection of presence and/or levels of these and other markers can be combined with the results from assay systems of the invention to provide a final result to the patient.

In other specific aspects, the results obtained in the assay systems of the invention are combined with the results obtained from ultrasound images, including but are not limited to: nuchal translucency (NT) thickness or edema, nuchal fold thickness, abnormality of the venous system (including the ductus venosus, the portal and hepatic veins and inferior vena cava), absent or hypoplastic nasal bone, femur length, humerus length, hyperechogenic bowel, renal pyelectasis, echogenic foci in the heart, fetal heart rate, and certain cardiac abnormalities. In specific aspects, the additional assessment of fetal abnormality is performed though shape analysis, such as described in U.S. Pat. Nos. 7,780,600 and 7,244,233. In a specific aspect, the additional assessment is based on the determination of landmarks based on images, as described in U.S. Pat. No. 7,343,190. Detection of these and other physical parameters can be combined with the results from assay systems of the invention to provide a final result to the patient.

Most screening markers and physical characteristics are known to vary with gestational age. To take account of this variation each marker level may be expressed as a multiple of the median level (MoM) for unaffected pregnancies of the same gestational age. Especially, for markers derived from ultrasound scans, crown-rump length (CRL) or biparietal diameter (BPD) measurement are alternative measures of gestational age. MoMs may be adjusted in a known way to take account of factors which are known to affect marker levels, such as maternal weight, ethnic group, diabetic status and the number of fetuses carried.

Use of the above techniques can be performed at a single stage of pregnancy or obtained sequentially at two or more different stages of pregnancy. These marker levels can also be interpreted in combination with variables maternal such as maternal age, weight, ethnicity, etc. to derive a risk estimate. The estimation of risk is conducted using standard statistical techniques. For example, known methods are described in Wald N J et al., BMJ (1992); 305(6850):391-4; Wald N J et al (1988) BMJ 297:883-887 and in Royston P, Thompson S G Stat Med. (1992) 11(2):257-68.

### Detection of Maternal Carrier Status

The assay systems of the present invention are useful not only in determining aneuploidy and other chromosomal abnormalities in the fetus, they can also be used to determine the genetic status of the mother at various alleles depending on the genomic regions interrogated in the assay system. By detecting certain selected nucleic acid regions corresponding to alleles that may contain genetic alterations associated with heritable diseases, disorders, and predispositions, maternal contribution can be detected as well as detecting any genetic alterations that may directly affect the fetus. The assay systems of the invention can thus be used for determining the carrier status of the mother and/or the fetus by detecting genetic alterations within the maternal and/or fetal contribution of the cell free DNA in a maternal sample.

Determining the carrier status of a pregnant female may thus be of value in both the present pregnancy as well as any future pregnancies, as it is predictive of the statistical likelihood that a fetus will inherit the genetic mutation. The American College of Medical Genetics (ACMG) recommends carrier screening by genetic testing for all prospective parents for a number of Mendelian diseases, and the list of diseases that ACMG recommends testing is continually expanding as a function of new discoveries related to genetic diseases. Estimates of genetic load indicate that every human carries approximately 8 to 30 deleterious recessive alleles.

Presently, screening of prospective parents for carrier status of a genetic anomaly (e.g., a point mutation, a translocation, or other genetic alteration associated with one or more disorders) is selective based on relative risk factors, e.g., due to race, ethnicity, family history or other factors that places individuals at increased risk for particular conditions. The assay systems of the invention allow universal testing for a wide variety of genetic conditions for individuals of any ancestry as a matter of course in determining the genetic status of the fetus.

For example, in certain assay systems in which the maternal sample is subjected to interrogation for particular point mutations, indels, and the like associated with disease, the mother's carrier status for such genetic alterations can be determined at the same time the fetal DNA in the maternal sample is interrogated. The ratios of the numbers of mutations detected will allow determination of an affected fetus, a fetus carrying a disease allele, and a mother's carrier status. In some circumstances, the paternal carrier status may be determined as well, e.g., when the fetus is affected with a homozygous autosomal recessive disorder or an autosomal dominant disorder for which the mother is not a carrier. A heterozygous carrier will display the genetic alteration in approximately 50% of the interrogated alleles containing the genetic alteration.

Given the multiplexed nature of the assay systems of the invention, in certain aspects it may be beneficial to utilize the assay to detect other nucleic acids that could pose a risk to the health of the mother or fetus, or that can otherwise impact on clinical decisions about the treatment or prognostic outcome for a subject. For example, in certain maternal samples of interest, the immune suppression of the subject may increase the risk for the disease due to changes in the subject's immune system. Specifically, changes in immunity and physiology during pregnancy may make pregnant women more susceptible to or more severely affected by certain disorders.

The assay systems of the invention could thus include interrogation of nucleic acids that indicators of disease or other risk for maternal or fetal health. Such indicators include, but are not limited to, genes associated with Rh status; mutations or polymorphisms associated with diseases such as diabetes, hyperlipidemia, hypercholesterolemia, blood disorders such as sickle cell anemia, hemophilia or thalassemia, cardiac conditions, etc.; somatic mutations or copy number variations associated with autoimmune disorders or malignancies (e.g., breast cancer), or any other health issue that may impact on the subject, and in particular on the clinical options that may be available in the treatment and/or prevention of health risks in a subject based on the outcome of the assay results.

Accordingly, as the preferred assay systems of the invention are highly multiplexed and able to interrogate hundreds or even thousands of nucleic acids within a maternal sample, in certain aspects it is desirable to interrogate the sample for nucleic acid markers within the maternal sample, e.g., nucleic acids associated with genetic or other health risk in the mother and/or fetus. Thus, in certain aspects, the assay systems provide detection of such nucleic acids in conjunction with the detection of chromosomal abnormalities and infectious agents within a maternal sample.

In specific aspects, the mother's carrier status can be determined for mutations associated with autosomal recessive disorders or other clinically relevant SNPs. The mother's carrier status can be determined by the ratio of alleles that comprise the mutation, which will comprise 50% of the maternally derived cell free DNA in a maternal sample. So, for example, when a maternal sample comprises 90% maternally derived DNA and 10% fetal DNA, the 50% of the maternally derived DNA would be 45% of the total DNA in a maternal sample.

Examples of mutations and SNPs that can be detected include but are not limited to those found in a database of genetic variants maintained by the US National Institutes of Health. See, e.g., Bhagwat M, Curr Protoc Bioinformatics. 2010 December; Chapter 1: Unit 1.19; Phillips C, Mol Biotechnol. 2007 January; 35(1):65-97. The curated records in dbSNP contain information that describes the sequence and location of genetic variants, and where available the frequency of alleles of those variants in different populations.

In other aspects, the mother's carrier status can be determined for mutations associated with autosomal dominant disorders, and in particular autosomal dominant alleles associated with diseases or disorders with an onset following reproductive age. In some aspects, the mother's carrier status can be determined for true autosomal dominant diseases such as, e.g., Huntington's disease. In other specific aspects, the mother's carrier status can be determined for mutations and/or SNPs associated with a predisposition for developing an adult disease or disorder, such as mutations in the BRCA1 and BRCA2 genes for breast and ovarian cancers. In yet other specific aspects, the mother's carrier status can be determined for mutations and/or SNPs associated with a poor prognosis in a disorder, e.g., the apoE4 allele of the apoE gene which is associated with an increased risk for and poor prognosis in Alzheimer's disease.

In particular aspects, the mother's carrier status can be determined for X-linked disorders. Carrier females who have only one copy of the mutation do not usually express the phenotype, although differences in X chromosome inactivation can lead to varying degrees of clinical expression in carrier females since some cells will express one X allele and some will express the other. The current estimate of sequenced X-linked genes is 499 and the total including vaguely defined traits is 983. See, e.g., the OMIM database, Johns Hopkins University. Such disorders include, but are not limited to, Hemophilia A, Hemophilia B, Duchenne muscular dystrophy, Becker's muscular dystrophy, Red-Green color blindness, X-linked ichthyosis, X-linked agammaglobulinemia (XLA); Glucose-6-phosphate dehydrogenase deficiency, Adrenoleukodystrophy, Alport syndrome; Androgen insensitivity syndrome, Barth syndrome; Centronuclear myopathy, Charcot-Marie-Tooth disease (CMTX2-3), Coffin-Lowry syndrome, Fabry disease, Hunter's Syndrome, Hypohidrotic ectodermal dysplasia, Kabuki syndrome, Kennedy disease, Lesch-Nyhan syndrome, Lowe Syndrome, Menkes disease, Nonsyndromic deafness, X-linked nonsyndromic deafness, Norrie disease, Occipital horn syndrome, Ornithine transcarbamylase deficiency, Siderius X-linked mental retardation syndrome (caused by mutations in the histone demethylase PHF8), Simpson-Golabi-Behmel syndrome; Spinal muscular atrophy (caused by a UBE1 gene mutation), Wiskott-Aldrich syndrome, X-linked Severe Combined Immunodeficiency (SCID), and X-linked sideroblastic anemia.

In yet other specific aspects, the mother's carrier status can be determined for chromosomal rearrangements such as translocations, duplications, or deletions. For example, the chromosomal translocations that may be detected in the mother include reciprocal translocations (also known as non-Robertsonian) and Robertsonian. This may be particularly helpful if the mother has a balanced translocation (in an even exchange of material in the mother with no genetic information extra or missing, and ideally full functionality) that may result in an unbalanced translocation (where the exchange of chromosome material is unequal resulting in extra or missing genes) in the fetus. In another example, chromosomal deletions such as the 22q11.2 deletion syndrome (which results in DiGeorge syndrome) can be detected.

In a preferred aspect, the mother's carrier status is determined in a maternal sample at the same time as chromosomal abnormality and infectious disease detection is performed on the same maternal sample. In a preferred aspect the analysis of maternal carrier status and fetal aneuploidy detection and infectious disease detection is performed at the same time in the same vessel. In a preferred aspect the analysis of maternal carrier status and fetal aneuploidy detection and infectious disease detection is performed using a highly multiplexed assay format such as disclosed in co-pending application U.S. Ser. No. 13/013,732, Barany et al, Oliphant et al or Willis et al. When the maternal carrier status is performed with fetal aneuploidy detection, maternal alleles are identified separate from paternally derived alleles in the fetus by looking for allele frequencies greater than 25%. The advantages of combining maternal carrier screening with aneuploidy detection include convenience, since only one blood draw is necessary, and cost, since the incremental costs of adding additional tests is much less than testing conditions separately.

While this invention is satisfied by aspects in many different forms, as described in detail in connection with preferred aspects of the invention, it is understood that the present disclosure is to be considered as exemplary of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. §112, ¶6.

What is claimed is:

1. An assay system for detection of the presence or absence of chromosomal abnormality and the presence or absence of an infectious agent in a maternal sample, comprising the steps of:
providing a maternal sample;
amplifying two or more selected nucleic acid regions from cell free nucleic acids corresponding to a first chromosome in the maternal sample;
amplifying two or more selected nucleic acid regions from cell free nucleic acids corresponding to a second chromosome in the maternal sample;
amplifying one or more selected nucleic acid regions corresponding to an infectious agent in the maternal sample;
detecting the amplified nucleic acid regions corresponding to the first and second chromosome;
quantifying the relative frequency of the selected nucleic acid regions from the first and second chromosomes;
comparing the relative frequency of the selected nucleic acid regions from the first and second chromosomes;
identifying the presence or absence of a chromosomal abnormality based on the compared relative frequencies of the first and second chromosomes;
detecting the amplified nucleic acid regions corresponding to the infectious agent; and
identifying the presence or absence of the infectious agent based on the detected amplified nucleic acid region corresponding to the infectious agent.

2. The assay system of claim 1, wherein the amplified nucleic acid regions corresponding to the first and second chromosomes are detected separately from the detection of the amplified nucleic acids corresponding to the infectious agent.

3. The assay system of claim 1, wherein the amplified nucleic acid regions corresponding to the first and second chromosomes and the amplified nucleic acids corresponding to the infectious agent are detected using a single detection method.

4. The assay system of claim 3, wherein the relative frequencies of the selected nucleic acid regions are individually quantified, and the relative frequencies of the individual nucleic acid regions are compared to determine the presence or absence of a chromosomal abnormality.

5. The assay system of claim 1, wherein the relative frequencies of the selected nucleic acid regions are individually quantified and summed by genomic region, and the summations compared to determine the presence or absence of a chromosomal abnormality.

6. The assay system of claim 1, wherein the quantified relative frequencies of the selected nucleic acid regions are normalized following detection and prior to quantification.

7. The assay system of claim 1, where the nucleic acid regions undergo a universal amplification.

8. The assay system of claim 1, where the nucleic acid regions are assayed in a single vessel.

9. The assay system of claim 1, where the nucleic acid regions are each counted an average of at least 500 times.

10. The assay system of claim 1, wherein the maternal sample is maternal blood, maternal plasma or maternal serum.

11. The assay system of claim 10, wherein the maternal sample is maternal plasma.

12. The assay system of claim 1, wherein the chromosomal abnormality is a fetal chromosomal abnormality.

13. An assay system for detection of the presence or absence of a chromosomal abnormality and the presence or absence of an infectious agent in a maternal sample, comprising the steps of:
providing a maternal sample;
isolating nucleic acids from the maternal sample;
amplifying two or more selected nucleic acid regions corresponding to a first chromosome from the isolated nucleic acids;
amplifying two or more selected nucleic acid regions corresponding to a second chromosome from the isolated nucleic acids;
amplifying one or more selected nucleic acid regions corresponding to an infectious agent from the isolated nucleic acids;
detecting the amplified nucleic acid regions;
quantifying the relative frequency of the selected nucleic acid regions from the first and second chromosomes;
comparing the relative frequency of the selected nucleic acid regions from the first and second chromosomes;
identifying the presence or absence of a chromosomal abnormality based on the compared relative frequencies of the first and second chromosome; and
identifying the presence or absence of the infectious agent based on the detected amplified nucleic acid region corresponding to the infectious agent.

14. The assay system of claim 13, wherein the amplified nucleic acid regions corresponding to the first and second chromosomes are detected separately from the detection of the amplified nucleic acids corresponding to the infectious agent.

15. The assay system of claim 13, wherein the amplified nucleic acid regions corresponding to the first and second chromosomes and the amplified nucleic acids corresponding to the infectious agent are detected using a single detection method.

16. The assay system of claim 13, wherein the relative frequencies of the selected nucleic acid regions are individually quantified, and the relative frequencies of the individual nucleic acid regions are compared to determine the presence or absence of a chromosomal abnormality.

17. The assay system of claim 13, wherein the relative frequencies of the selected nucleic acid regions are individually quantified and summed by genomic region and the summations compared to determine the presence or absence of a chromosomal abnormality.

18. The assay system of claim 13, wherein the quantified relative frequencies of the selected nucleic acid regions are normalized following detection and prior to quantification.

19. The assay system of claim 13, wherein the cell free nucleic acids from the maternal sample comprise cell free DNA.

20. The assay system of claim 13, wherein the cell free nucleic acids isolated from the maternal sample comprise RNA.

21. The assay system of claim 20, wherein the RNA is converted to DNA prior to amplification of the selected nucleic acid regions.

22. The assay system of claim 13, wherein the cell free nucleic acids isolated from the maternal sample comprise both DNA and RNA, and wherein the RNA is converted to DNA prior to the amplification of the selected nucleic acid region.

23. The assay system of claim 13, where the nucleic acid regions undergo a universal amplification.

24. The assay system of claim 13, where the nucleic acid regions are assayed in a single vessel.

25. The assay system of claim 13, where the nucleic acid regions are each counted an average of at least 500 times.

26. The assay system of claim 13, wherein the maternal sample is maternal blood, maternal plasma or maternal serum.

27. The assay system of claim 26, wherein the maternal sample is maternal plasma.

28. The assay system of claim 13, wherein the chromosomal abnormality is a fetal chromosomal abnormality.

29. An assay system that provides simultaneous detection of the presence or absence of a chromosomal abnormality and the presence or absence of an infectious agent using nucleic acids from a single maternal sample.

30. The assay system of claim 29, wherein the nucleic acids are detected in a single vessel.

31. The assay system of claim 29, wherein the nucleic acids are isolated from the maternal sample prior to detection.

32. The assay system of claim 31, wherein the nucleic acids for detection of the chromosomal abnormality are isolated separately from the isolation of the nucleic acids for the detection of the infectious agent.

33. The assay system of claim 32, wherein the isolated nucleic acids are combined prior to detection.

34. An assay system for detection of the presence or absence of chromosomal abnormality and the presence or absence of an infectious agent in a maternal sample, comprising the steps of:
providing a maternal sample;
amplifying two or more selected nucleic acid regions from cell free nucleic acids corresponding to a first chromosome in the maternal sample;
amplifying two or more selected nucleic acid regions from cell free nucleic acids corresponding to a second chromosome in the maternal sample;
amplifying one or more selected nucleic acid regions corresponding to an infectious agent in the maternal sample;
detecting the amplified nucleic acid regions corresponding to the first and second chromosome;
quantifying the relative frequency of the selected nucleic acid regions from the first and second chromosomes;
comparing the relative frequency of the selected nucleic acid regions from the first and second chromosomes;
identifying the presence or absence of a chromosomal abnormality based on the compared relative frequencies of the first and second chromosomes;
detecting the amplified nucleic acid regions corresponding to the infectious agent; and
identifying the presence or absence of the infectious agent based on the detected amplified nucleic acid region corresponding to the infectious agent,
wherein the amplified nucleic acid regions corresponding to the first and second chromosomes and the amplified nucleic acids corresponding to the infectious agent are detected using a single detection method.

35. The method of claim 34, wherein the amplified nucleic acid regions corresponding to the first and second chromosomes and the amplified nucleic acids corresponding to the infectious agent are detected in the same detection step.
